(19) United States
(12) Patent Application Publication
(10) Pub. No.: US 2025/0066372 A1
(43) Pub. Date: Feb. 27, 2025

(54) HETEROCYCLIC COMPOUNDS, COMPOSITIONS THEREOF, AND METHODS OF TREATMENT THEREWITH

(71) Applicant: BeiGene, Ltd., Camana Bay (KY)

(72) Inventors: Chao YU, Beijing (CN); Hanzi SUN, Beijing (CN); Ce WANG, Beijing (CN); Zhiwei WANG, Suzhou (CN)

(73) Assignee: BeiGene, Ltd., Camana Bay (KY)

(21) Appl. No.: 18/934,390

(22) Filed: Nov. 1, 2024

Related U.S. Application Data
(63) Continuation of application No. PCT/CN23/92047, filed on May 4, 2023.

(30) Foreign Application Priority Data
May 5, 2022 (WO) PCT/CN22/90877

(51) Int. Cl. C07D 487/04; A61K 31/519

(52) U.S. Cl. CPC C07D 487/04; A61K 31/519

(57) ABSTRACT

Provided herein are compounds having the following structure:

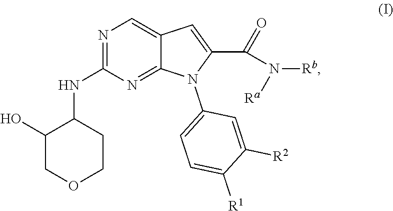

or a pharmaceutically acceptable salt, tautomer, stereoisomer, or enantiomer thereof, wherein the substituents are as defined herein compositions comprising an effective amount of a compound, and methods for inhibiting activity of cyclin-dependent kinases.

### CROSS-REFERENCE TO RELATED APPLICATIONS

This application is a continuation of International Application No. PCT/CN2023/092047, filed May 4, 2023, which claims priority to International Application No. PCT/CN2022/090877, filed May 5, 2022. The disclosures of the aforementioned applications are incorporated herein by reference in their entireties.

### FIELD

Provided herein are compounds useful for inhibiting cyclin-dependent kinases, methods of using the compounds disclosed herein for treating cancer, and a pharmaceutical composition comprising the same.

### BACKGROUND

Human kinase is a large group of enzymes that add phosphate groups (PO₄3−) to other molecules in human body [1. FASEB J. 1995 May; 9(8):576-96. 2. Enzyme Res. 2011; 2011: 794089.]. There are more than 500 kinase-encoding genes exist in the human genome and their substrates including proteins, lipids, and nucleic acids [3. Cell Signal. 2004 September; 16(9):983-9. 4. Cell. 2017 Aug. 10; 170(4):605-635.]. Kinase mis-regulation is identified in many diseases including cancer, autoimmunity, neurological disorders, diabetes and cardiovascular disease. For example, the mutated kinases can become constitutively active and thus cause diverse cellular anomalies, leading to cancer initiation or growth. Using small molecular inhibitors to inhibit kinase activity is proved to be a successful method to treat cancer and other disease [5. Expert Rev Anticancer Ther. 2018 December; 18(12):1249-1270.]. Up to now, there are more than 70 kinase inhibitors have been approved by FDA, EMA or CDE as drugs [6. Nat Rev Drug Discov. 2018 May; 17(5):353-377.].

Protein kinase family take a majority fraction of the kinase superfamily. For protein targets, protein kinases can phosphorylate the amino acids including serine, threonine, tyrosine and histidine. [7. Science. 2002 Dec. 6; 298(5600):1912-34.] Protein kinases play a major role in cellular activation processes, through reversible phosphorylation and dephosphorylation of proteins, by the antagonistic action of kinases and phosphatases, is an important component of cell signaling because the phosphorylated and unphosphorylated states of the target protein can have different levels of activity. [8. Biochimie. 2014 December; 107 Pt B:167-87. 9. Clin Transl Oncol. 2006 March; 8(3):153-60.] Different protein kinases including EGFR, BTK, ALK, JAK, P13K and CDK are proved to be good targets for cancer drug development.

Excessively activated cell cycle is a common feature of human cancer [10. Nat Rev Cancer. 2009 March; 9(3):153-66.]. While cyclins are among the most important core cell cycle regulators. There are four basic cyclin types found in humans including G1 cyclins, G1/S cyclins, S cyclins and M cyclins. To drive the cell cycle forward, a cyclin must activate or inactivate many target proteins inside of the cell. And these cyclins drive the events of the cell cycle majorly by partnering with a family of enzymes called the cyclin-dependent kinases (Cdks). Cdk kinase itself is inactive, but binding with a cyclin can activates it, making the CDK/cyclin complex a functional holoenzyme and allowing it to modify target proteins [11. Orphanet J Rare Dis. 2020 Aug. 6; 15(1):203. 12. J Mol Biol. 1999 Apr. 16; 287(5):821-8.]. There are 26 serine/threonine protein kinases that form a CDK and CDK-like branch of the CMGC subfamily of the human kinome; of these, 21 are classified as CDKs. Among all the currently identified CDKs, CDK1, CDK2, CDK4 and CDK6 are considered as the direct modulate of cell cycle majorly by phosphorylating and inactivate RB protein and release E2F transcription factors, and E2F downstream pathway is critical in regulating the initiation of DNA replication. And CDK4/6 is essential for G1 early initiation and G1/S transition. [13. Cell Death Differ. 1998 February; 5(2):132-40. 14. Oncogene. 2016 Sep. 15; 35(37):4829-35.] CDK4/6 related pathway is commonly deregulated in many different cancer types such as breast cancer, lung cancer and pancreatic cancer. And there are 4 approved CDK4/6 inhibitors including palbociclib, ribociclib, abemaciclib and trilaciclib which have been approved by FDA or CDE to be used as either single agent or combo with endocrine therapy to treat HR+, Her2− breast cancer. This approach shows good efficacy in clinic while CDK4/6 inhibitors more or less lead to hemopoietic toxicity like neutropenia and leukopenia which highly limit the clinical application of CDK4/6 inhibitors. And emerging data indicating inhibition of CDK6/Cyclin D3 may cause the clinical observed hematologic toxicity [15. Cell. 2004 Aug. 20; 118(4):493-504. 16. Haematologica. 2021 Oct. 1; 106(10):2624-2632.] while CDK4/Cyclin D1 is the oncogenic driver in different cancers [17. Nat Commun. 2019 Dec. 20; 10(1):5817. 18. 18. Cancer Cell. 2006 January; 9(1):23-32.]. Develop a CDK4 selective inhibitor might lead to advantages including improved efficacy, mitigated hematologic toxicity and expanding clinical usage in many cancers including but not limited to breast cancer, lung cancer, pancreatic cancer, prostate cancer, bone cancer, liver cancer and endometrial cancer.

There remains great need to develop a CDK4 selective inhibitor since the protein structure of CDK4 and CDK4 share very high homology. Most of previously reported compounds are CDK4/6 dual inhibitors. Here we report compounds with high CDK4 selectivity all other kinases including CDK6, which potentially lead to better efficacy, improved toxicity profile and potential to overcome resistance mechanisms, and the like.

Citation or identification of any reference in this section is not to be construed as an admission that the reference is prior art to the present application.

### SUMMARY

Provided herein are compounds having the following formula (I):

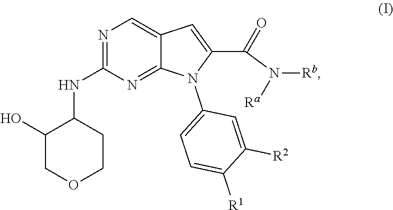

or a pharmaceutically acceptable salt, tautomer, stereoisomer, or enantiomer thereof,
wherein:
each of R^{a} and R^{b} is, independently, hydrogen or substituted or unsubstituted C₁₋₈ alkyl;
or R^{a} and ^{b}R, together with the nitrogen to which R^{a} and R^{b} connect, form a substituted or unsubstituted non-aromatic heterocyclyl;
each of R¹ and R² is, independently, hydrogen, halogen, substituted or unsubstituted C₁₋₈ alkyl, substituted or unsubstituted cycloalkyl, substituted or unsubstituted non-aromatic heterocyclyl, substituted or unsubstituted saturated cycloalkylalkyl, substituted or unsubstituted non-aromatic heterocyclylalkyl; or
R¹ and R², together with the atoms to which R¹ and R² connect, form a substituted or unsubstituted cycloalkyl, or substituted or unsubstituted non-aromatic heterocyclyl.

Provided here is a pharmaceutical composition comprising an effective amount of a compound provided herein, or a pharmaceutically acceptable salt, tautomer, isotopologue, stereoisomer, or prodrug thereof, and a pharmaceutically acceptable carrier, excipient or vehicle.

Provided here is a method of inhibiting activity of CDK kinases in a cell, comprising contacting said cell with an effective amount of a compound provided herein, or a pharmaceutically acceptable salt, tautomer, isotopologue, stereoisomer, or prodrug thereof. In one embodiment, the CDK kinase is CDK4 kinase.

Provided here is a method for the treatment or prevention of a cancer responsive to CDK activity, the methods comprising administering to a subject in need thereof an effective amount of a compound provided herein. In one embodiment, the CDK is CDK4 kinase.

### DETAILED DESCRIPTION

### Definitions

The term “CDK” as used herein refers to cyclin-dependent kinase protein, a member of the protein kinase family that regulates the cell cycle. Known CDKs include CDK1, CDK2, CDK3, CDK4, CDK5, CDK6, CDK7, CDK8, CDK9, CDK10, and CDK11. A “CDK inhibitor” is a substance that (i) interacts directly with a CDK, e.g., by binding to a CDK, and (ii) reduces the expression or activity of the CDK. The term also includes naturally occurring variants of CDK, including CDK1, CDK2, CDK3, CDK4, CDK5, CDK6, CDK7, CDK8, CDK9, CDK10, and CDK11.

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 20 (or 0.2 degree 20) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; dialkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracylic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{a})₂, —C(O)NH(R^{a}) or —C(O)NH₂, wherein each R^{a} is independently a substituted or unsubstituted alkyl, cycloalkyl, aryl, aralkyl, heterocyclyl or heterocyclyl group as defined herein.

An “acylamino” group is a radical of the formula: —NHC(O)(R^{a}) or —N(alkyl)C(O)(R^{a}), wherein each alkyl and R^{a} are independently as defined above.

A “sulfonylamino” group is a radical of the formula: —NHSO₂(R^{a}) or —N(alkyl)SO₂(R^{a}), wherein each alkyl and R^{a} are defined above.

A “urea” group is a radical of the formula: —N(alkyl)C(O)N(R^{a})₂, —N(alkyl)C(O)NH(R^{a}), —N(alkyl)C(O)NH₂, —NHC(O)N(R^{a})₂, —NHC(O)NH(R^{a}), or —NH(CO)NHR^{a}, wherein each alkyl and Rare independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

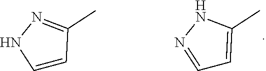

As readily understood by one skilled in the art, a wide variety of functional groups and other structures may exhibit tautomerism and all tautomers of compounds of formula (I) are within the scope of the present invention.

It should also be noted the compounds can contain unnatural proportions of atomic isotopes at one or more of the atoms. For example, the compounds may be radiolabeled with radioactive isotopes, such as for example tritium (H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In some embodiments, “treating” means an alleviation, in whole or in part, of a disorder, disease or condition, or a slowing, or halting of further progression or worsening of those symptoms. In another embodiment, “treating” means and alleviation, in whole or in part, of a disorder, disease or condition, or symptoms associated with a condition, wherein the condition is treatable or preventable by inhibition of CDK, e.g., CDK4.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the condition is a condition, treatable or preventable by inhibition of CDK, e.g., CDK4.

The term “effective amount” in connection with a compound means an amount capable of treating or preventing a disorder, disease or condition, or symptoms thereof, disclosed herein.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

### Compounds

The present embodiments can be understood more fully by reference to the detailed description and examples, which are intended to exemplify non-limiting embodiments.

Aspect 1: Provided herein is a compound of formula (I):

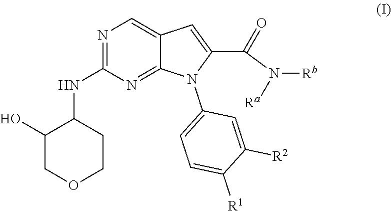

or a pharmaceutically acceptable salt, tautomer, stereoisomer, or enantiomer thereof,
wherein:
each of R^{a} and R^{b} is, independently, hydrogen or substituted or unsubstituted C₁₋₈ alkyl;
or R^{a} and R^{b}, together with the nitrogen to which R^{a} and R^{b} connect, form a substituted or unsubstituted non-aromatic heterocyclyl;
each of R¹ and R² is, independently, hydrogen, halogen, substituted or unsubstituted C₁₋₈ alkyl, substituted or unsubstituted cycloalkyl, substituted or unsubstituted non-aromatic heterocyclyl, substituted or unsubstituted saturated cycloalkylalkyl, substituted or unsubstituted non-aromatic heterocyclylalkyl; or
R¹ and R², together with the atoms to which R¹ and R² connect, form a substituted or unsubstituted cycloalkyl, or substituted or unsubstituted non-aromatic heterocyclyl.

In one embodiment, the compound is a compound of formula (Ia):

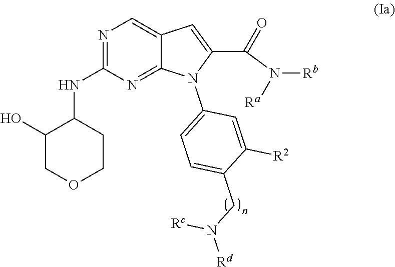

or a pharmaceutically acceptable salt, tautomer, stereoisomer, or enantiomer thereof,
each of R^{c} and R^{d} is, independently, hydrogen or substituted or unsubstituted C₁₋₈ alkyl;
or R^{c} and R^{d}, together with the nitrogen to which R^{c} and R^{d} connect, form a substituted or unsubstituted non-aromatic heterocyclyl; and n is 1, 2 or 3.

In one embodiment, the compound is a compound of formula (Ib):

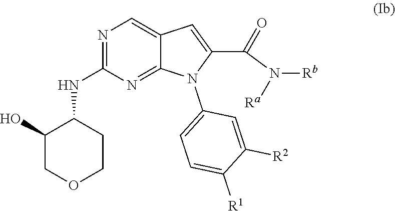

or a pharmaceutically acceptable salt, tautomer, stereoisomer, or enantiomer thereof.

In one preferred embodiment, R¹ is C₁₋₈ alkyl substituted with amino, alkylamino or dialkylamino.

Aspect 2: Provided herein is a compound is a compound of formula (II):

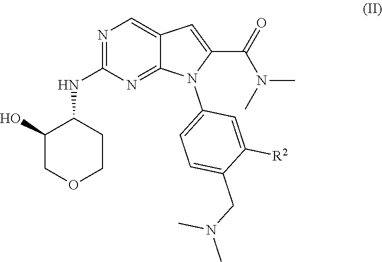

or a pharmaceutically acceptable salt, tautomer, stereoisomer, or enantiomer thereof,
wherein:
R² is hydrogen, halogen, substituted or unsubstituted C₁₋₈ alkyl, or substituted or unsubstituted cycloalkyl.

In one embodiment, R² is hydrogen, F, Cl, —CF₃, or cyclopropyl.

In one embodiment, R² is Cl.

In one embodiment, R² is hydrogen.

In one embodiment, R² is cyclopropyl.

In one embodiment, R² is F.

In one embodiment, R² is —CF₃.

Aspect 3: Provided herein is a compound is a compound of formula (III):

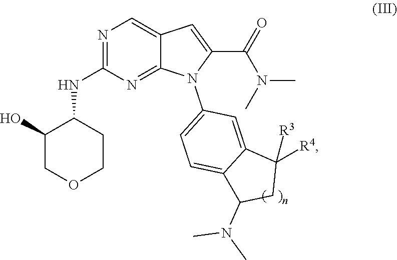

or a pharmaceutically acceptable salt, tautomer, stereoisomer, or enantiomer thereof,
wherein:
each of R³ and R⁴ is, independently, hydrogen, halogen, substituted or unsubstituted C₁₋₈ alkyl; or
R³ and R⁴, together with the atom to which R³ and R⁴ connect, form a substituted or unsubstituted cycloalkyl, or substituted or unsubstituted non-aromatic heterocyclyl; and
n is 1, 2, or 3.

In one embodiment, R³ and R⁴ are F; and n is 2.

In one embodiment, R³ and R⁴, together with the atom to which R³ and R⁴ connect, form cyclopropyl; and n is 1.

In one embodiment, R³ and R⁴, together with the atom which R³ and R⁴ connect, form cyclopropyl; and n is 2.

In one embodiment, R³ and R⁴ are H; and n is 1.

In one embodiment, R³ and R⁴ are H; and n is 2.

Aspect 4: Provided herein is a compound of formula (IV):

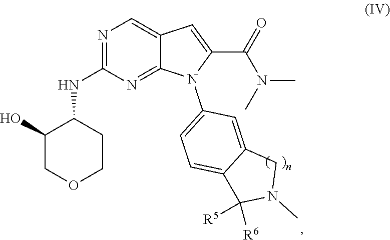

or a pharmaceutically acceptable salt, tautomer, stereoisomer, or enantiomer thereof,
wherein:
each of R⁵ and R⁶ is, independently, hydrogen, halogen, substituted or unsubstituted C₁₋₈ alkyl; or
R⁵ and R⁶, together with the atom which R¹ and R⁶ connect, form a substituted or unsubstituted cycloalkyl, or substituted or unsubstituted non-aromatic heterocyclyl; and
n is 1, 2, or 3.

In one embodiment, R⁵ and R⁶ are H; and n is 2.

In one embodiment, R¹ is methyl; and R² is H.

In one embodiment, the compound is selected from Table 1.

Aspect 5: Provided herein is a pharmaceutical composition comprising an effective amount of a compound provided herein, or a pharmaceutically acceptable salt, tautomer, isotopologue, stereoisomer, or prodrug thereof, and a pharmaceutically acceptable carrier, excipient or vehicle.

Provided herein is a method of inhibiting activity of cyclin-dependent kinases in a cell, comprising contacting said cell with an effective amount of a compound provided herein, or a pharmaceutically acceptable salt, tautomer, isotopologue, stereoisomer, or prodrug thereof.

In one embodiment, the cyclin-dependent kinase is CDK4.

In one embodiment, the compound is selective for CDK4 over CDK6.

In one embodiment, the compound is selective for CDK4 over CDK1, CDK2, CDK3, CDK5, CDK6, CDK7, CDK8, CDK9, CDK10, or CDK11.

In one embodiment, the compound is at least 20-fold selective for CDK4 over CDK6. In one embodiment, the compound is at least 50-fold selective for CDK4 over CDK6. In one embodiment, the compound is at least 100-fold selective for CDK4 over CDK6.

In one embodiment, provided herein is a method for the treatment or prevention of CDK mediated disorder, the methods comprising administering to a subject in need thereof an effective amount of a compound provided herein. In one embodiment, the CDK is CDK4.

In one embodiment, provided herein is a method for the treatment or prevention of a cancer responsive to CDK activity, the methods comprising administering to a subject in need thereof an effective amount of a compound provided herein. In one embodiment, the CDK is CDK4.

### Methods for Making Compounds

The Compounds can be made using conventional organic syntheses and commercially available starting materials. By way of example and not limitation, Compounds of formula (I), formula (II), formula (III), and formula (IV) can be prepared as outlined in Schemes 1-4 shown below as well as in the examples set forth herein. It should be noted that one skilled in the art would know how to modify the procedures set forth in the illustrative schemes and examples to arrive at the desired products. Common protecting groups may be used to prevent certain functional groups from undergoing undesired reaction. Exemplary protecting groups are described in “Protective Groups in Organic Synthesis”, 4^{th} Edition, P. G. M. Wuts; T. W. Greene, John Wiley, 2007, and references cited therein.

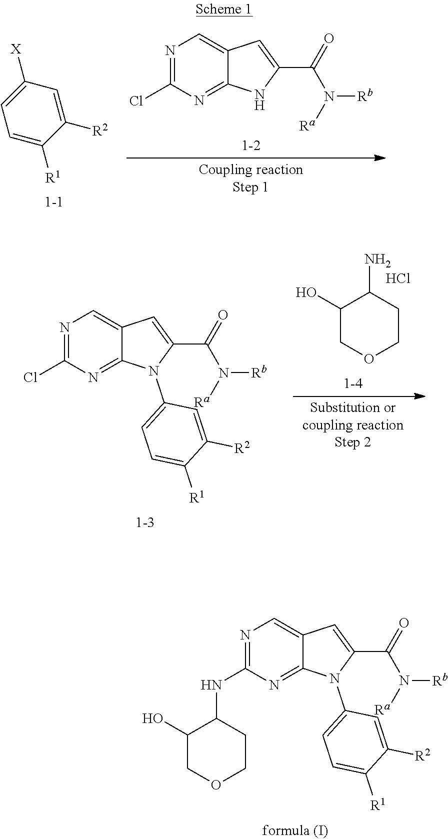

As shown in Scheme 1, in some embodiments, provided herein are methods for preparing the compounds defined as formula (I). Compound 1-1 (X may be halogen, boronic acid, or boronic ester) is converted into compound 1-3 under Chan-Lam or Ullman coupling reaction conditions (e.g., Cu(OAc)2, pyridine, dioxane, oxygen); then compound 1-3 is converted to the compound defined as formula (I) under substitution or coupling reaction conditions (e.g., Palladium catalyst, Cs₂CO₃, dioxane, etc.).

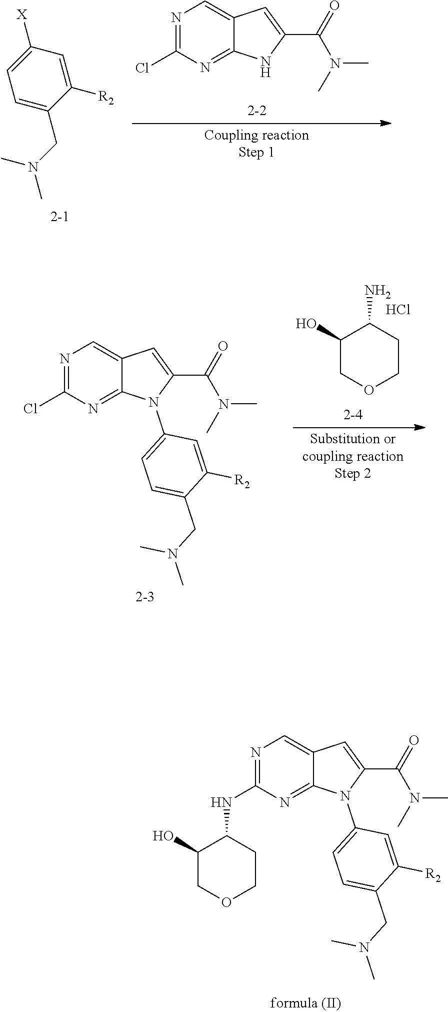

As shown in Scheme 2, in some embodiments, provided herein are methods for preparing the compounds defined as formula (II). Compound 2-1 (X may be halogen, boronic acid, or boronic ester) is converted into compound 2-3 under Chan-Lam or Ullman coupling reaction conditions (e.g., Cu(OAc)2, pyridine, dioxane, oxygen); then compound 2-3 is converted to the compound defined as formula (II) under substitution or coupling reaction conditions (e.g., Palladium catalyst, Cs₂CO₃, dioxane, etc.).

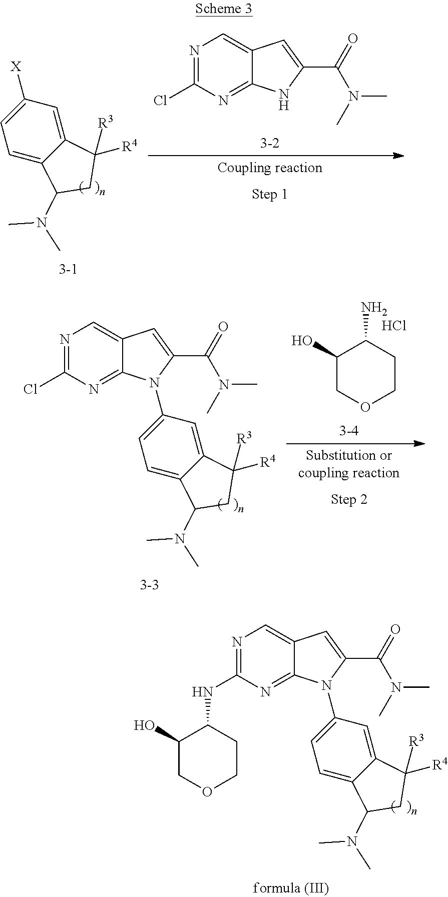

As shown in Scheme 3, in some embodiments, provided herein are methods for preparing the compounds defined as formula (III). Compound 3-1 (X may be halogen, boronic acid, or boronic ester) is converted into compound 3-3 under Chan-Lam or Ullman coupling reaction conditions (e.g., Cu(OAc)2, pyridine, dioxane, oxygen); then compound 3-3 is converted to the compound defined as formula (III) under substitution or coupling reaction conditions (e.g., Palladium catalyst, Cs₂CO₃, dioxane, etc.).

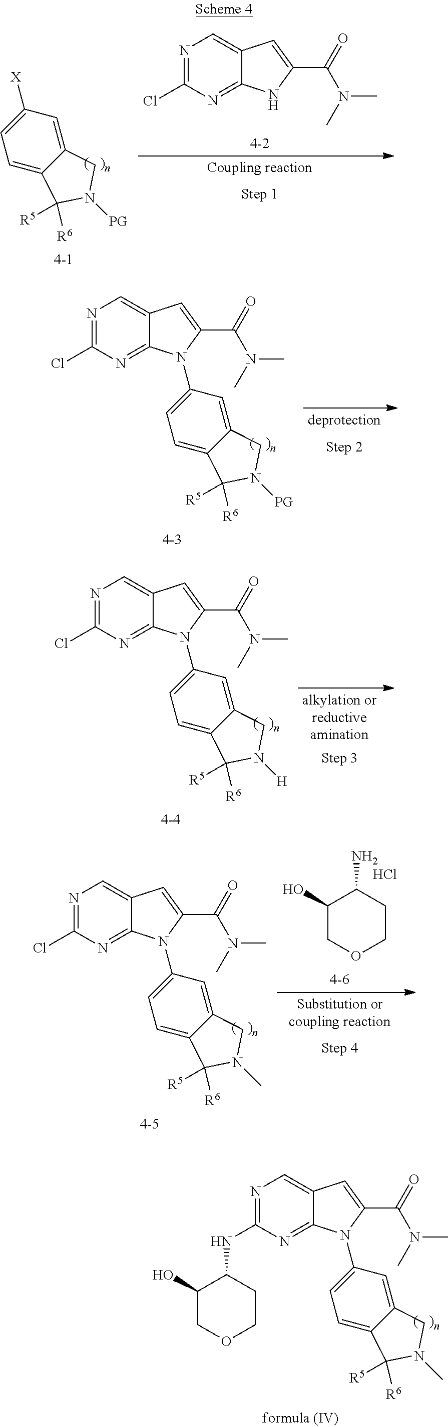

As shown in Scheme 4, in some embodiments, provided herein are methods for preparing the compounds defined as formula (IV). Compound 4-1 (X may be halogen, boronic acid, or boronic ester) is converted into compound 4-3 under Chan-Lam or Ullman coupling reaction conditions (e.g., Cu(OAc)2, pyridine, dioxane, oxygen); then compound 4-3 is converted to compound 4-4 under deprotection conditions (e.g. TFA to deprotect Boc group when PG is Boc); compound 4-4 further undergoes alkylation or reductive amination to give compound 4-5; compound 4-5 is converted to the compound defined as formula (IV) under substitution or coupling reaction conditions (e.g., Palladium catalyst, Cs₂CO₃, dioxane, etc.).

The present embodiments can be understood more fully by reference to the detailed description and examples, which are intended to exemplify non-limiting embodiments.

### Examples

The examples below are intended to be purely exemplary and should not be considered to be limiting in any way. Unless otherwise indicated, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400 or Varian instrument operating at 300 MHz, 400 MHz, or 500 MHz. ¹H-NMR spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₂CO: 2.05) as the reference standard. Coupling constants were given in hertz. Peaks were reported as singlet (s), doublet (d), triplet (t), quartet (q), quintet (p), sextet (h), septet (hept), multiplet (m), or a combination thereof; br stands for broad. LC/MS data was recorded by using Agilent 1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 19.1.

In the following examples, the following abbreviations are used:
AcOH Acetic acid
Aq. Aqueous
BINAP 2,2′-bis(diphenylphosphino)-1,1′-binaphthalene
Brine Saturated aqueous sodium chloride solution
Bn Benzyl
BnBr Benzyl Bromide
Boc Tert-butoxycarbonyl
CH₂Cl₂ or DCM Dichloromethane
CAN Cerium(IV) ammonium nitrate (cericammonium nitrate)
DAST Diethylaminosulfur trifluoride
DMF N,N-Dimethylformamide
Dppf 1,1′-bis(diphenylphosphino)ferrocene
DBU 1,8-diazabicyclo[5.4.0]undec-7-ene
DHP 3,4-Dihydro-2H-pyran
DIEA or DIPEA N,N-diisopropylethylamine
DMAP 4-N,N-dimethylaminopyridine
DMB (2,4-dimethoxyphenyl)methanamine
Dess-Martin/DMP Dess-Martin Periodinane
DMF N,N-dimethylformamide
DMF-DMA N,N-Dimethylformamide dimethyl acetal purum
DMSO Dimethyl sulfoxide
DMEDA Dimethyl Ethylene Diamine
EDCI 1-Ethyl-3-(3-dimethylaminopropyl)carbodiimide hydrochloride
EtOAc or EA Ethyl acetate
EtOH Ethanol
Et₃SiH Triethyl silhydride
Et₂O or ether Diethyl ether
g Grams
h or hr Hour
HATU O-(7-Azabenzotriazol-1-yl)-N,N,N′,N′-tetramethyluronium hexafluorophosphate
Hex Hexane
HCl Hydrochloric acid
HMDS Hexamethyldisilazane
HOBT 1-Hydroxybenzotriazole
HPLC High-performance liquid chromatography
IBX 2-Iodylbenzoic acid
i-PrOH Isopropyl alcohol
LCMS Liquid chromatography-mass spectrometry
LDA Lithium diisopropylamide
LiHMDS Lithium Bis(trimethylsilyl)amide
K₂OsO₄·H2O Potassium osmate(VI) dihydrate
mg Milligrams
mL Milliliters
mmol Millimole
MeCN Acetonitrile
MeOH Methanol
Min Minutes
ms or MS Mass spectrum
m-CPBA 2-chloranylbenzenecarboperoxoic acid
MPLC Medium Pressure Liquid Chromatography
Na₂SO₄ Sodium sulfate
NaBH(OAc)₃/STAB Sodium triacetyl borohydride
NaHMDS Sodium bis(trimethylsilyl)amide
NBS N-Bromosuccinimide
NCS N-Chlorosuccinimide
NMO 4-Methylmorpholine N-oxide
NMP N-Methyl Pyrrolidone
PE petroleum ether
PMB (4-methoxyphenyl)methanamine
POCl₃ phosphorous oxychloride
PyBOP Benzotriazol-1-yl-oxytripyrrolidinophosphonium
hexafluorophosphate
PddppfCl₂ [1,1′-Bis(diphenylphosphino)ferrocene]dichloropalladium(II)
Pd₂(dba)₃ Tris(dibenzylideneacetone)dipalladium
Prep Preparative
PTSA 4-Methylbenzenesulfonic acid
Rt or rt Room temperature
sat. Saturated
SEMCl (2-(Chloromethoxy)ethyl)trimethylsilane
TBSCl tert-Butyldimethylsilyl chloride
TEA/Et3N triethylamine
t-BuOK Potassium tert-butoxide
t-BuONa Sodium tert-butoxide
T₃P n-Propylphosphonic cyclic anhydride
TMSCN Trimethylsilyl cyanide
TFA Trifluoroacetic acid
TFAA Trifluoroacetic anhydride
THF Tetrahydrofuran
TLC thin layer chromatography
tBuXPhospd-G3 Methanesulfonato(2-di-t-butylphosphino-2′,4′,6′-tri-i-propyl-1,1′-biphenyl)(2′-amino-1,1′-biphenyl-2-yl)palladium(II)
tBuXPhos 2-Di-tert-butylphosphino-2′,4′,6′-triisopropylbiphenyl
UHP Urea hydrogen peroxide
Ml Microliters
XantPhos 4,5-Bis(diphenylphosphino)-9,9-dimethylxanthene
XPhos 2-Dicyclohexylphosphino-2′,4′,6′-triisopropylbiphenyl
4CzIPN (4r,6r)-2,4,5,6-tetra(9H-carbazol-9-yl)isophthalonitrile

### Compound Synthesis

### Example 1: 7-cyclopentyl-2-(((3S,4R)-3-hydroxytetrahydro-2H-pyran-4-yl)amino)-N,N-dimethyl-7H-pyrrolo[2,3-d]pyrimidine-6-carboxamide

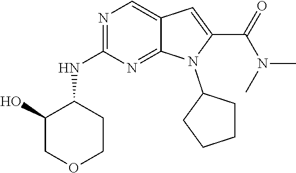 [SMILES: CN(C)C(=O)c1cc2cnc(N[C@@H]3CCOC[C@H]3O)nc2n1C1CCCC1]

### Synthetic Route

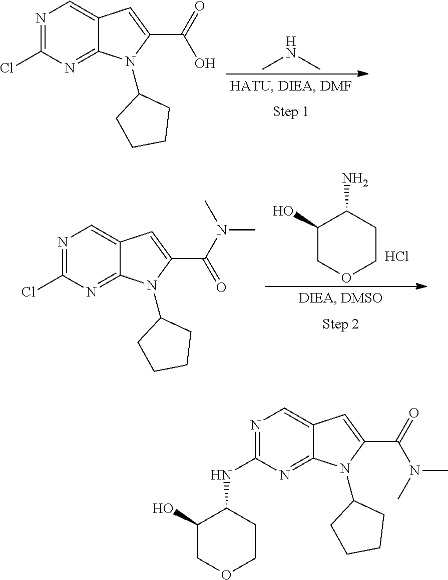

### Step 1: 2-chloro-7-cyclopentyl-N,N-dimethyl-7H-pyrrolo[2,3-d]pyrimidine-6-carboxamide

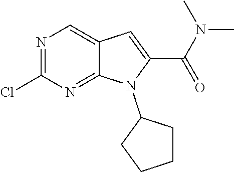 [SMILES: CN(C)C(=O)c1cc2cnc(Cl)nc2n1C1CCCC1]

Into a 50-mL round-bottom flask was placed 2-chloro-7-cyclopentyl-7H-pyrrolo[2,3-d]pyrimidine-6-carboxylic acid (300 mg, 1.13 mmol), DMF (20 mL) were added. Dimethylamine hydrochloride (137 mg, 1.70 mmol), DIEA (291 mg, 2.26 mmol), and HATU (515 mg, 1.36 mmol) were then added. The resulting solution was stirred for 2 hrs at room temperature. The reaction was quenched with water (30 mL) and extracted with EtOAc (25 mL*3). Combined organic layer was washed with brine (25 mL*3), dried over sodium sulfate and concentrated under reduced pressure. The crude product was purified by Prep-TLC to give the title product (360 mg). MS (ESI, m/e) [M+H]⁺ 293.1.

### Step 2: 7-cyclopentyl-2-(((3S,4R)-3-hydroxytetrahydro-2H-pyran-4-yl)amino)-N,N-dimethyl-7H-pyrrolo[2,3-d]pyrimidine-6-carboxamide

Into a 50-mL round-bottom flask were placed 2-chloro-7-cyclopentyl-N,N-dimethyl-7H-pyrrolo[2,3-d]pyrimidine-6-carboxamide (300 mg, 1.02 mmol), (3S,4R)-4-aminotetrahydro-2H-pyran-3-ol hydrochloride (468 mg, 3.06 mmol), DIEA (329 mg, 2.55 mmol), and DMSO (5 mL). The resulting solution was stirred for 1 h at 120° C. The reaction was quenched with water (20 mL). The resulting mixture was extracted with EA (15 mL*3).

The resulting mixture was washed with brine (15 mL*3). The organic layers
were concentrated under reduced pressure. The crude product was purified by Prep-HPLC to give the title product (57.2 mg). ¹H NMR (400 MHz, DMSO-d₆) δ 8.56 (s, 1H), 6.79-6.70 (m, 1H), 6.47 (s, 1H), 5.00-4.93 (m, 1H), 4.78-4.62 (m, 1H), 3.88-3.80 (m, 2H), 3.74 (s, 1H), 3.62-3.47 (m, 1H), 3.40-3.33 (m, 1H), 3.14-2.96 (m, 7H), 2.41-2.26 (m, 2H), 2.17-2.05 (m, 1H), 2.05-1.88 (m, 4H), 1.72-1.56 (m, 2H), 1.55-1.40 (m, 1H). MS (ESI, m/e) [M+H]⁺ 374.0.

### Example 2: 2-(((3S,4R)-3-hydroxytetrahydro-2H-pyran-4-yl)amino)-N,N-dimethyl-7-(p-tolyl)-7H-pyrrolo[2,3-d]pyrimidine-6-carboxamide

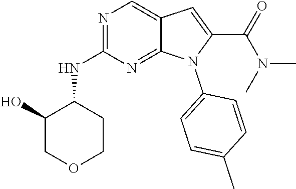 [SMILES: Cc1ccc(-n2c(C(=O)N(C)C)cc3cnc(N[C@@H]4CCOC[C@H]4O)nc32)cc1]

### Synthetic Route

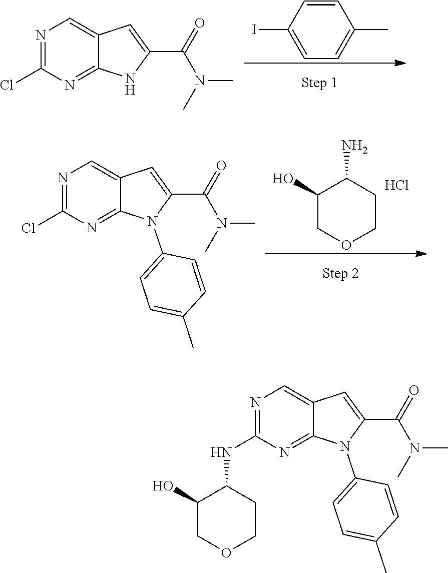

### Step 1: 2-chloro-N,N-dimethyl-7-(p-tolyl)-7H-pyrrolo[2,3-d]pyrimidine-6-carboxamide

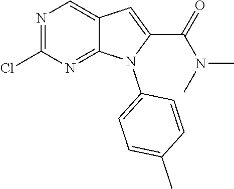 [SMILES: Cc1ccc(-n2c(C(=O)N(C)C)cc3cnc(Cl)nc32)cc1]

A mixture of 2-chloro-N,N-dimethyl-7H-pyrrolo[2,3-d]pyrimidine-6-carboxamide (200 mg, 0.893 mmol), CuI (33.8 mg, 0.178 mmol), (S)-Proline (10.2 mg, 0.089 mmol) and K₂CO₃ (246 mg, 1.785 mmol) in DMSO (5 mL) was stirred for 16 hrs at 90° C. The resulting mixture was extracted with EtOAc (2×200 mL). The combined organic layers were washed with brine (2×100 mL), dried over anhydrous Na₂SO₄. After filtration, the filtrate was concentrated under reduced pressure. The resulting mixture was concentrated under vacuum. The residue was purified by Prep-TLC (PE/EA=3:1) to afford the title product (70 mg). MS (ESI, m/e) [M+H]⁺ 315.2.

### Step 2: 2-(((3S,4R)-3-hydroxytetrahydro-2H-pyran-4-yl)amino)-N,N-dimethyl-7-(p-tolyl)-7H-pyrrolo[2,3-d]pyrimidine-6-carboxamide

A mixture of 2-chloro-N,N-dimethyl-7-(p-tolyl)-7H-pyrrolo[2,3-d]pyrimidine-6-carboxamide (70 mg, 0.222 mmol), (3S,4R)-4-aminotetrahydro-2H-pyran-3-ol hydrochloride (51 mg, 0.333 mmol), Cs₂CO₃ (145 mg, 0444 mmol) and Pd-PEPPSI-IPentCl 2-methylpyridine (o-picoline) (15.6 mg, 0.02 mmol) in dioxane (1 mL) was stirred for 16 hrs at 100° C. The resulting mixture was concentrated under vacuum. The crude product (10 mg) was purified by Prep-HPLC to give the title product (38 mg). ¹H NMR (300 MHz, CDCl₃) δ 8.57 (s, 1H), 7.36-7.24 (m, 4H), 6.70 (s, 1H), 4.14-3.93 (m, 2H), 3.92-3.76 (m, 1H), 3.69-3.57 (m, 1H), 3.51-3.38 (m, 1H), 3.26-3.13 (m, 1H), 3.06-2.83 (m, 6H), 2.44 (s, 3H), 2.05-1.95 (m, 1H), 1.83-1.64 (m, 1H). MS (ESI, m/e) [M+H]⁺ 396.1.

### Example 3: 7-(4-((dimethylamino)methyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

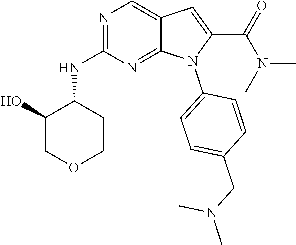 [SMILES: CN(C)Cc1ccc(-n2c(C(=O)N(C)C)cc3cnc(N[C@@H]4CCOC[C@H]4O)nc32)cc1]

### Synthetic Route

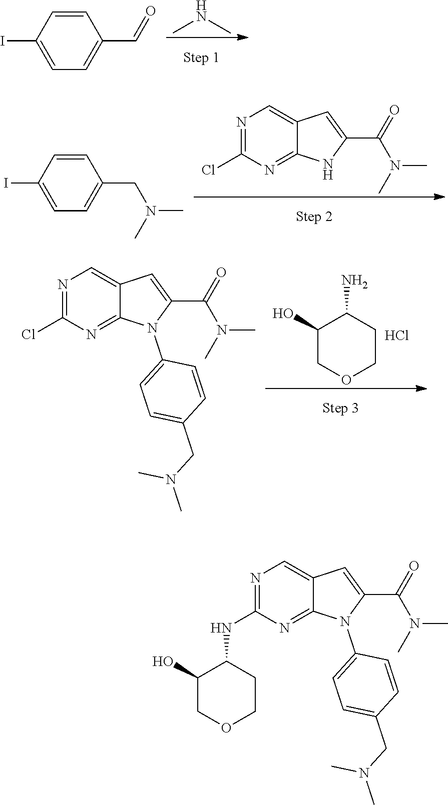

### Step 1: 1-(4-iodophenyl)-N,N-dimethylmethanamine

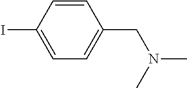

To a solution of 4-iodobenzaldehyde (1.5 g, 6.49 mmol) in DCE (10 mL) and MeOH (10 mL) was added dimethylamine (6.46 mL, 12.9 mmol, 2 M in THF) at room temperature. After stirring for 3 hrs, STAB (2.75 g, 13.0 mmol) was added. The resulting solution was stirred overnight at room temperature. The resulting mixture was quenched with H₂O and extracted with EtOAc. The organic layer was concentrated. The residue was purified with silica gel column (PE:EtOAc=100:0˜50:50). This resulted in the title product (1.0 g). MS (ESI, m/e) [M+H]⁺ 261.9.

### Step 2: 2-chloro-7-(4-((dimethylamino)methyl)phenyl)-N,N-dimethyl-7H-pyrrolo[2,3-d]pyrimidine-6-carboxamide

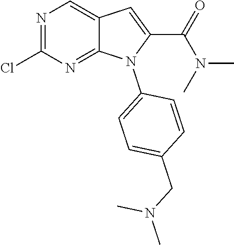 [SMILES: CN(C)Cc1ccc(-n2c(C(=O)N(C)C)cc3cnc(Cl)nc32)cc1]

Into a 40-mL vial, were placed 1-(4-iodophenyl)-N,N-dimethylmethanamine (500 mg, 1.92 mmol), 2-chloro-N,N-dimethyl-7H-pyrrolo[2,3-d]pyrimidine-6-carboxamide (431 mg, 1.92 mmol), (S)-Proline (22 mg, 0.192 mmol), K₂CO₃ (528 mg, 3.83 mmol), CuI (36 mg, 0.192 mmol), and DMSO (5 mL). The resulting mixture was stirred for 16 hrs at 90° C. The mixture was quenched with H₂O and extracted with EtOAc. The organic layer was concentrated. The residue was purified by Prep-TLC to give the title product (80 mg). MS (ESI, m/e) [M+H]⁺ 358.1.

### Step 3: 7-(4-((dimethylamino)methyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

Into a 20-mL vial, were placed 2-chloro-7-(4-((dimethylamino)methyl)phenyl)-N,N-dimethyl-7H-pyrrolo[2,3-d]pyrimidine-6-carboxamide (80 mg, 0.22 mmol), (3S,4R)-4-aminotetrahydro-2H-pyran-3-ol hydrochloride (51.4 mg, 0.34 mmol), Cs₂CO₃ (219 mg, 0.67 mmol), [1,3-bis[2,6-bis(1-ethylpropyl)phenyl]-4,5-dichloro-1,3-dihydro-2H-imidazol-2-ylidene]dichloro(2-methylpyridine)Palladium (18 mg, 0.02 mmol) and dioxane (1 mL). The mixture was stirred overnight at 90° C. The resulting mixture was concentrated. The residue was applied onto a silica gel column with DCM/MeOH (6:1) to afford the crude product. The crude product was purified by Prep-HPLC to give the title product (9.3 mg). ¹H NMR (300 MHz, CD₃OD) δ 8.79 (s, 1H), 7.74-7.65 (m, 2H), 7.65-7.54 (m, 2H), 7.04 (s, 1H), 4.89 (s, 2H), 4.41-3.79 (m, 3H), 3.66-3.58 (m, 1H), 3.39-3.35 (m, 1H), 3.18-3.12 (m, 4H), 3.08 (s, 3H), 2.91 (s, 6H), 2.07-1.98 (m, 1H), 1.80-1.60 (m, 1H). MS (ESI, m/e) [M+H]⁺ 439.2.

### Example 4: 7-(3-cyclopropyl-4-((dimethylamino)methyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

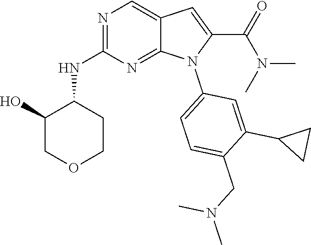 [SMILES: CN(C)Cc1ccc(-n2c(C(=O)N(C)C)cc3cnc(N[C@@H]4CCOC[C@H]4O)nc32)cc1C1CC1]

### Synthetic Route

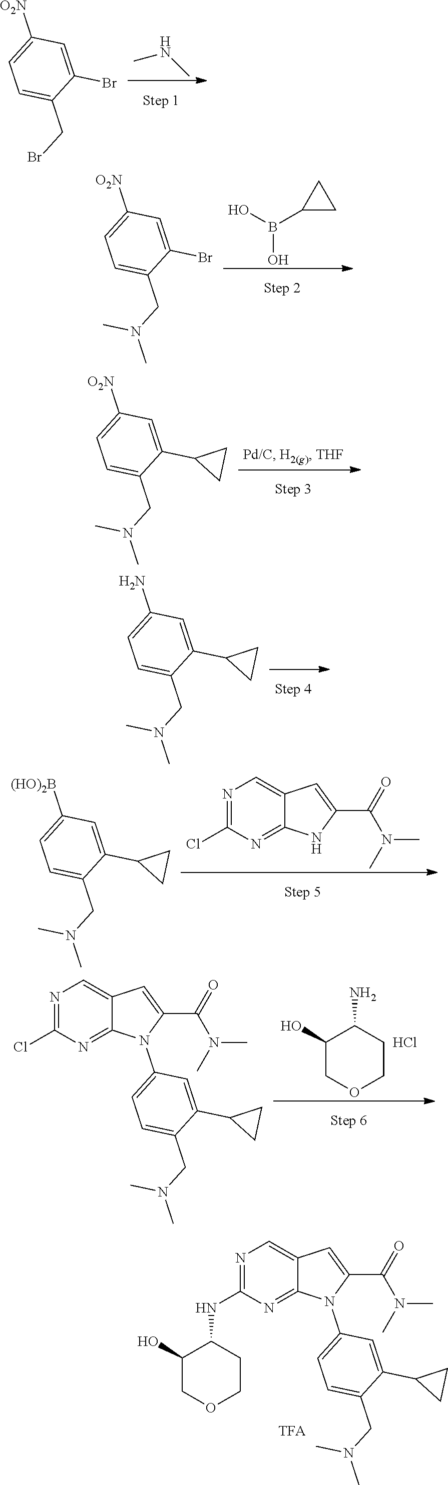

### Step 1: 1-(2-bromo-4-nitrophenyl)-N,N-dimethylmethanamine

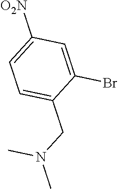

A solution of 2-bromo-1-(bromomethyl)-4-nitrobenzene (5.20 g, 17.6 mmol) and dimethylamine (1.99 g, 44.1 mmol) in MeOH (52 mL) was stirred for 3 hrs at room temperature. The resulting mixture was concentrated under reduced pressure. The residue was purified by silica gel column chromatography to give the title product (4.5 g). MS (ESI, m/e) [M+H]⁺ 259.0.

### Step 2: 1-(2-cyclopropyl-4-nitrophenyl)-N,N-dimethylmethanamine

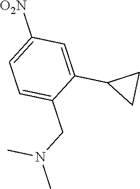 [SMILES: CN(C)Cc1ccc([N+](=O)[O-])cc1C1CC1]

A solution of 1-(2-bromo-4-nitrophenyl)-N,N-dimethylmethanamine (3.0 g, 11.6 mmol), cyclopropylboronic acid (2.98 g, 34.7 mmol), Pd(OAc)₂ (0.26 g, 1.2 mmol), tricyclohexylphosphane (0.65 g, 2.3 mmol) and K₃PO₄ (11.06 g, 52.1 mmol) in toluene/H₂O (30 mL/10 mL) was stirred overnight at 110° C. The resulting mixture was filtered and the filtered cake was washed with EtOAc (3×20 mL). The filtrate was concentrated under reduced pressure. The resulting mixture was extracted with EtOAc (3×20 mL). The resulting mixture was concentrated under vacuum. The residue was purified by silica gel column chromatography to give the title product (1.3 g). MS (ESI, m/e) [M+H]⁺ 221.0.

### Step 3: 3-cyclopropyl-4-((dimethylamino)methyl)aniline

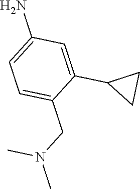

A solution of 1-(2-cyclopropyl-4-nitrophenyl)-N,N-dimethylmethanamine (1.3 g, 5.90 mmol) in THF (13 mL) was stirred overnight at room temperature under hydrogen atmosphere. The resulting mixture was filtered and the filtered cake was washed with THF (3×20 mL). The filtrate was concentrated under reduced pressure. The crude product (1.2 g) was used in the next step directly without further purification. MS (ESI, m/e) [M+H]⁺ 191.1.

### Step 4: (3-cyclopropyl-4-((dimethylamino)methyl)phenyl)boronic acid

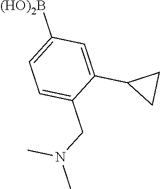 [SMILES: CN(C)Cc1ccc(B(O)O)cc1C1CC1]

A mixture of 3-cyclopropyl-4-((dimethylamino)methyl)aniline (1.1 g, 5.8 mmol) and NaNO₂ (0.40 g, 5.8 mmol) in HCl(3M)/MeOH/H₂O (7 mL/14 mL/7 mL) was stirred for 30 min at 0° C. To the above mixture was added diboronic acid (1.55 g, 17.3 mmol) at 0° C. The resulting mixture was stirred for 1 h at room temperature. The resulting mixture was filtered and the filter cake was washed with MeOH (3×15 mL). The filtrate was concentrated under reduced pressure. The residue was purified by C18 column chromatographyto give the title product (400 mg). MS (ESI, m/e) [M+H]⁺ 220.0.

### Step 5: 2-chloro-7-(3-cyclopropyl-4-((dimethylamino)methyl)phenyl)-N,N-dimethyl-7H-pyrrolo[2,3-d]pyrimidine-6-carboxamide

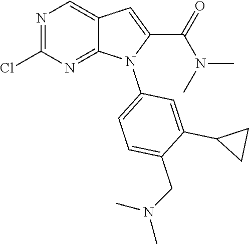 [SMILES: CN(C)Cc1ccc(-n2c(C(=O)N(C)C)cc3cnc(Cl)nc32)cc1C1CC1]

A solution of (3-cyclopropyl-4-((dimethylamino)methyl)phenyl)boronic acid (350 mg, 1.60 mmol), Cu(OAc)₂ (435 mg, 2.40 mmol), TEA (485 mg, 4.80 mmol) and 2-chloro-N,N-dimethyl-7H-pyrrolo[2,3-d]pyrimidine-6-carboxamide (359 mg, 1.60 mmol) in 1,4-dioxane (5 mL) was stirred for 16 hrs at 80° C. under oxygen atmosphere. The resulting mixture was filtered and the filter cake was washed with MeOH (3×10 mL). The filtrate was concentrated under reduced pressure. The residue was purified by Prep-TLC (CH₂Cl₂/MeOH=10:1) to give the title product (80 mg). MS (ESI, m/e) [M+H]⁺ 398.1.

### Step 6: 7-(3-cyclopropyl-4-((dimethylamino)methyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

A mixture of 2-chloro-7-(3-cyclopropyl-4-((dimethylamino)methyl)phenyl)-N,N-dimethyl-7H-pyrrolo[2,3-d]pyrimidine-6-carboxamide (80 mg, 0.20 mmol), Cs₂CO₃ (196.5 mg, 0.6 mmol), [1,3-bis[2,6-bis(1-ethylpropyl)phenyl]-4,5-dichloro-1,3-dihydro-2H-imidazol-2-ylidene]dichloro(2-methylpyridine)Palladium (17 mg, 0.02 mmol) and (3S,4R)-4-aminooxan-3-ol (35.3 mg, 0.30 mmol) in 1,4-dioxane was stirred overnight at 100° C. The resulting mixture was filtered and the filter cake was washed with MeOH (3×5 mL). The filtrate was concentrated under reduced pressure. The crude product was purified by Prep-HPLC to give the title product (6 mg). ¹H NMR (300 MHz, CD₃OD) δ 8.74 (s, 1H), 7.63-7.57 (m, 1H), 7.38-7.30 (m, 1H), 7.30-7.21 (m, 1H), 6.96 (s, 1H), 4.63 (s, 2H), 4.02-3.78 (m, 3H), 3.68-3.53 (m, 1H), 3.47-3.35 (m, 1H), 3.20-3.06 (m, 4H), 2.98 (s, 9H), 2.31-1.98 (m, 2H), 1.76-1.55 (m, 1H), 1.20-1.10 (m, 2H), 0.92-0.77 (m, 2H). MS (ESI, m/e) [M+H]⁺ 479.3.

### Example 5: 7-(4-((dimethylamino)methyl)-3-fluorophenyl)-2-(((3S,4R)-3-hydroxytetrahydro-2H-pyran-4-yl)amino)-N,N-dimethyl-7H-pyrrolo[2,3-d]pyrimidine-6-carboxamide

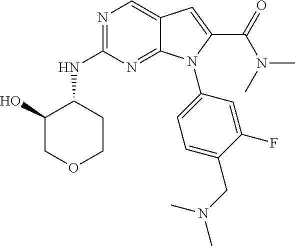 [SMILES: CN(C)Cc1ccc(-n2c(C(=O)N(C)C)cc3cnc(N[C@@H]4CCOC[C@H]4O)nc32)cc1F]

### Synthetic Route

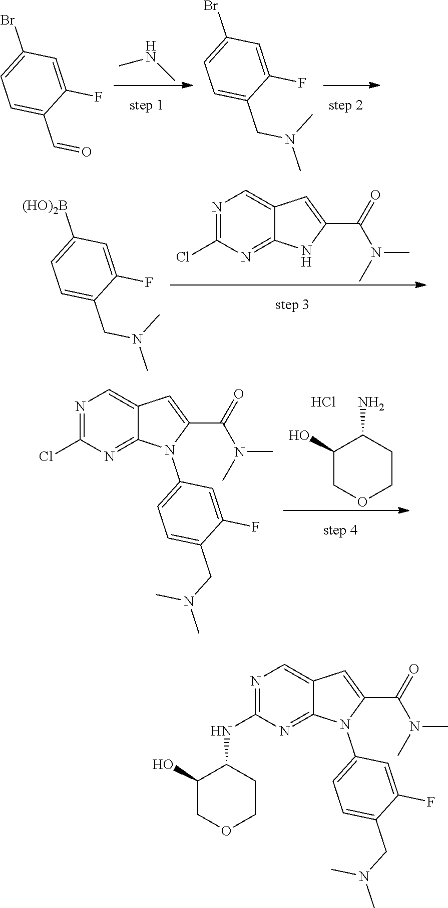

### Step 1: 1-(4-bromo-2-fluorophenyl)-N,N-dimethylmethanamine

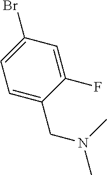

Into a 250-mL round-bottom flask were added 4-bromo-2-fluorobenzaldehyde (3.0 g, 15 mmol), DCE (50 mL) and dimethylamine (29.5 mL, 29.5 mmol, 1M in THF) at room temperature. After stirring for 1 h at room temperature, STAB (6.26 g, 29.5 mmol) was added. The resulting mixture was stirred overnight at room temperature. The reaction was quenched with NH₄Cl (aq.) at room temperature. The resulting mixture was extracted with EtOAc (2×200 mL). The combined organic layers were washed with brine (2×50 mL), and dried over anhydrous Na₂SO₄. After filtration, the filtrate was concentrated under reduced pressure. The residue was purified by silica gel column chromatographyto give the title product (1.2 g). MS (ESI, m/e) [M+H]⁺ 232.1.

### Step 2: (4-((dimethylamino)methyl)-3-fluorophenyl)boronic acid

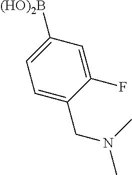

To a mixture of 1-(4-bromo-2-fluorophenyl)-N,N-dimethylmethanamine (1.0 g, 4.3 mmol) and THF (10 mL) was added N-butyllithium (5.19 mL, 12.9 mmol, 2.5M in THF) at −78° C. The reaction mixture was stirred at −78° C. for 30 min. Then, a solution of triisopropyl borate (2.03 g, 10.77 mmol) in THF (2 mL) was added dropwise and the mixture was stirred for another 1 h. The reaction was quenched with NH₄Cl (aq), and the resulting mixture was concentrated under reduced pressure. The residue was purified by reverse flash chromatography to give the title product (300 mg). MS (ESI, m/e) [M+H]⁺ 198.2.

### Step 3: 2-chloro-7-(4-((dimethylamino)methyl)-3-fluorophenyl)-N,N-dimethyl-7H-pyrrolo[2,3-d]pyrimidine-6-carboxamide

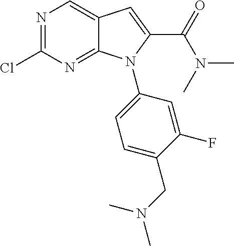 [SMILES: CN(C)Cc1ccc(-n2c(C(=O)N(C)C)cc3cnc(Cl)nc32)cc1F]

Into a 20-mL bottom flask were placed (4-((dimethylamino)methyl)-3-fluorophenyl)boronic acid (300 mg, 1.52 mmol), 2-chloro-N,N-dimethyl-7H-pyrrolo[2,3-d]pyrimidine-6-carboxamide, pyridine (602.2 mg, 7.61 mmol), Copper acetate (415 mg, 2.28 mmol), dioxane (10 mL) and 4A MS (100 mg) at room temperature. The resulting mixture was stirred for overnight at 80° C. under oxygen atmosphere. The residue was purified by silica gel column chromatography to give the title product (160 mg). MS (ESI, m/e) [M+H]⁺ 376.2.

### Step 4: 7-(4-((dimethylamino)methyl)-3-fluorophenyl)-2-(((3S,4R)-3-hydroxytetrahydro-2H-pyran-4-yl)amino)-N,N-dimethyl-7H-pyrrolo [2,3-d]pyrimidine-6-carboxamide

Into a 10-mL round-bottom flask were added 2-chloro-7-(4-((dimethylamino)methyl)-3-fluorophenyl)-N,N-dimethyl-7H-pyrrolo[2,3-d]pyrimidine-6-carboxamide (150 mg, 0.39 mmol), (3S,4R)-4-aminotetrahydro-2H-pyran-3-ol hydrochloride (70.1 mg, 0.59 mmol), Cs₂CO₃ (390.1 mg, 1.19 mmol), [1,3-bis[2,6-bis(1-ethylpropyl)phenyl]-4,5-dichloro-1,3-dihydro-2H-imidazol-2-ylidene]dichloro(2-methylpyridine)Palladium (33.5 mg, 0.04 mmol) and dioxane (5 mL) at room temperature. The resulting mixture was stirred 100° C. for overnight. The residue product was purified by reverse phase flash to give the title product (13.7 mg). ¹H NMR (400 MHz, CD₃OD) δ 8.74 (s, 1H), 7.74-7.65 (m, 1H), 7.62-7.53 (m, 1H), 7.42-7.34 (m, 1H), 7.02 (s, 1H), 4.48 (s, 2H), 3.97-3.80 (m, 3H), 3.67-3.56 (m, 1H), 3.45-3.36 (m, 1H), 3.26 (s, 3H), 3.17-3.09 (m, 1H), 3.03 (s, 3H), 2.95 (s, 6H), 2.12-2.02 (m, 1H), 1.73-1.60 (m, 1H). MS (ESI, m/e) [M+H]⁺ 457.1.

### Example 6: 7-(3-chloro-4-((dimethylamino)methyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

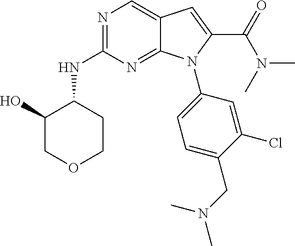 [SMILES: CN(C)Cc1ccc(-n2c(C(=O)N(C)C)cc3cnc(N[C@@H]4CCOC[C@H]4O)nc32)cc1Cl]

### Synthetic Route

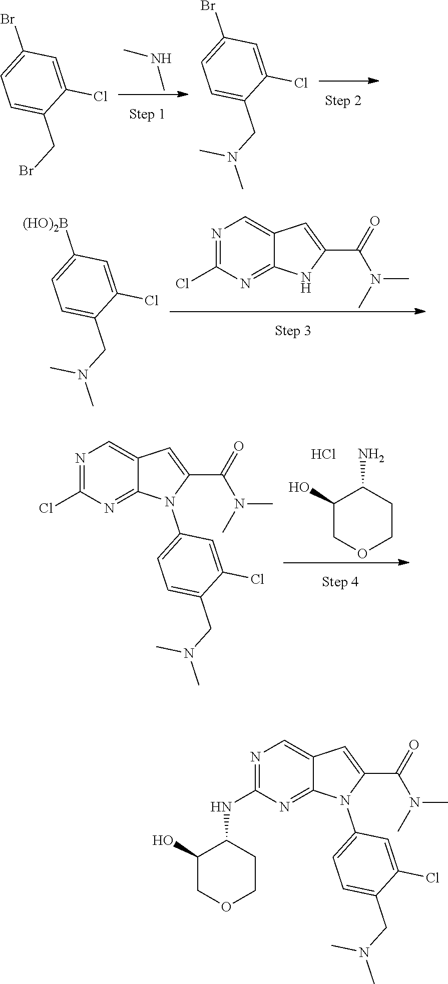

### Step 1: 1-(4-bromo-2-chlorophenyl)-N,N-dimethylmethanamine

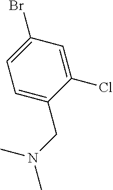

A solution of 4-bromo-1-(bromomethyl)-2-chlorobenzene (2.0 g, 7.0 mmol) and dimethylamine (0.63 g, 14 mmol) in MeOH was stirred for 5 hrs at room temperature. The resulting mixture was concentrated under reduced pressure. The residue was purified by silica gel column chromatography to afford the title product (1.2 g). MS (ESI, m/e) [M+H]⁺ 248.0.

### Step 2: (3-chloro-4-((dimethylamino)methyl)phenyl)boronic Acid

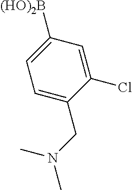

A solution of 1-(4-bromo-2-chlorophenyl)-N,N-dimethylmethanamine (1.0 g, 4.0 mmol) in THF was treated with butyllithium (0.77 g, 12 mmol) for 1 h at −78° C., followed by the addition of triisopropyl borate (1.89 g, 10.0 mmol) dropwise at −78° C. The resulting mixture was stirred for 2 hrs at room temperature. The resulting mixture was concentrated under reduced pressure. The residue was purified by C18 column chromatography to afford the title product (500 mg). MS (ESI, m/e) [M+H]⁺ 214.0.

### Step 3: 2-chloro-7-(3-chloro-4-((dimethylamino)methyl)phenyl)-N,N-dimethyl-7H-pyrrolo[2,3-d]pyrimidine-6-carboxamide

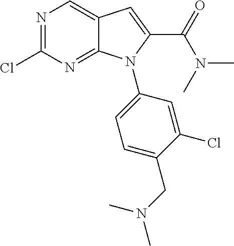 [SMILES: CN(C)Cc1ccc(-n2c(C(=O)N(C)C)cc3cnc(Cl)nc32)cc1Cl]

A solution of (3-chloro-4-((dimethylamino)methyl)phenyl)boronic acid (400 mg, 1.87 mmol), Cu(OAc)₂ (510.5 mg, 2.81 mmol), TEA (379.2 mg, 3.74 mmol) and 2-chloro-N,N-dimethyl-7H-pyrrolo[2,3-d]pyrimidine-6-carboxamide (294.6 mg, 1.31 mmol) in 1,4-dioxane was stirred for 16 hrs at 80° C. under oxygen atmosphere. The resulting mixture was filtered and the filter cake was washed with MeOH (3×10 mL). The filtrate was concentrated under reduced pressure. The residue was purified by silica gel column chromatography to give the title product (130 mg). MS (ESI, m/e) [M+H]⁺ 392.0.

### Step 4: 7-(3-chloro-4-((dimethylamino)methyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

A solution of 2-chloro-7-(3-chloro-4-((dimethylamino)methyl)phenyl)-N,N-dimethyl-7H-pyrrolo[2,3-d]pyrimidine-6-carboxamide (70 mg, 0.18 mmol), Cs₂CO₃ (174.4 mg, 0.53 mmol) and (3S,4R)-4-aminooxan-3-ol (31.36 mg, 0.26 mmol) in 1,4-dioxane was stirred overnight at 100° C. The resulting mixture was filtered, and the filter cake was washed with MeOH (3×5 mL). The filtrate was concentrated under reduced pressure. The crude product was purified by Prep-HPLC to give the title product (12.3 mg). ¹H NMR (400 MHz, DMSO-d₆) δ 8.68 (s, 1H), 7.59-7.51 (m, 1H), 7.32-7.24 (m, 1H), 6.98 (s, 1H), 6.80 (s, 1H), 4.96-4.87 (m, 1H), 3.85-3.76 (m, 2H), 3.60-3.44 (m, 3H), 3.15-2.78 (m, 8H), 2.23 (s, 6H), 2.12-1.98 (m, 1H), 1.53-1.38 (m, 1H). MS (ESI, m/e) [M+H]⁺ 473.2.

### Example 7: 7-(4-((dimethylamino)methyl)-3-(trifluoromethyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

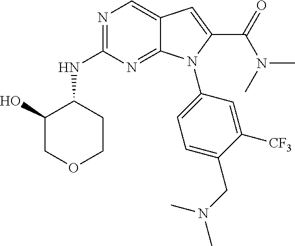 [SMILES: CN(C)Cc1ccc(-n2c(C(=O)N(C)C)cc3cnc(N[C@@H]4CCOC[C@H]4O)nc32)cc1C(F)(F)F]

### Synthetic Route

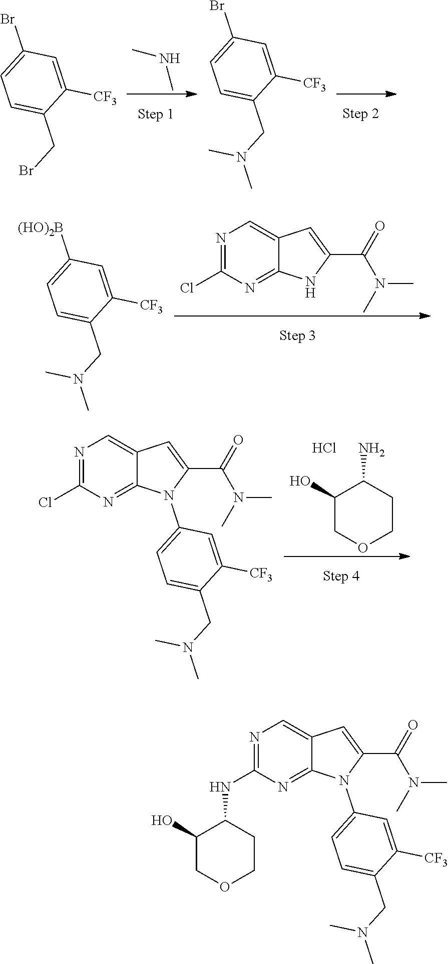

### Step 1: 1-(4-bromo-2-(trifluoromethyl)phenyl)-N,N-dimethylmethanamine

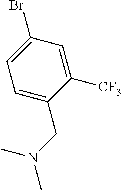

Into a 250-mL round-bottom flask were added MeOH (60 mL), 4-bromo-1-(bromomethyl)-2-(trifluoromethyl)benzene (3.7 g, 12 mmol) and dimethylamine (11.9 mL, 23.8 mmol, 2M in THF) at room temperature. The resulting mixture was stirred for 5 hrs at room temperature. The resulting mixture was concentrated under vacuum. The residue was purified by reverse phase HPLC to afford the title product (2.0 g). MS (ESI, m/e) [M+H]⁺ 282.0.

### Step 2: (4-((dimethylamino)methyl)-3-(trifluoromethyl)phenyl)boronic Acid

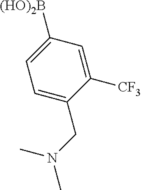 [SMILES: CN(C)Cc1ccc(B(O)O)cc1C(F)(F)F]

In a 50-mL round bottom flask, to a solution of 1-(4-bromo-2-(trifluoromethyl)phenyl)-N,N-dimethylmethanamine (1.80 g, 6.38 mmol) in THF (20 mL) was added dropwise n-butyllithium solution (3.3 mL, 8.29 mmol, 2.5M in hexane) at −78° C. The reaction mixture was stirred for 60 min. Then, a solution of triisopropyl borate (1.9 g, 10.2 mmol) in THF (4 mL) was added dropwise and the mixture was stirred for another 60 min at room temperature. The reaction was quenched with MeOH (50 mL) and the mixture was concentrated under vacuum to yield a crude product (4.0 g). The residue was purified by reverse phase HPLC to afford the title product (0.80 g). MS (ESI, m/e) [M+H]⁺ 248.0.

### Step 3: 2-chloro-7-(4-((dimethylamino)methyl)-3-(trifluoromethyl)phenyl)-N,N-dimethyl-7H-pyrrolo [2,3-d]pyrimidine-6-carboxamide

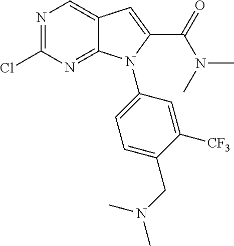 [SMILES: CN(C)Cc1ccc(-n2c(C(=O)N(C)C)cc3cnc(Cl)nc32)cc1C(F)(F)F]

Into a 25 mL vial were added dioxane (4 mL), (4-((dimethylamino)methyl)-3-(trifluoromethyl)phenyl)boronic acid (0.5 g, 2.02 mmol), 2-chloro-N,N-dimethyl-7H-pyrrolo[2,3-d]pyrimidine-6-carboxamide (450 mg, 2.02 mmol), TEA (0.61 g, 6.1 mmol) and Cu(OAc)₂ (0.56 g, 3.03 mmol) at room temperature. The resulting mixture was stirred for 4 hrs at 80° C. under oxygen atmosphere. The residue was purified by Prep-TLC (PE/EA=1:1) to afford the title product (200 mg). MS (ESI, m/e) [M+H]⁺ 426.1.

### Step 4: 7-(4-((dimethylamino)methyl)-3-(trifluoromethyl)phenyl)-2-(((3S,4R)-3-hydroxytetrahydro-2H-pyran-4-yl)amino)-N,N-dimethyl-7H-pyrrolo[2,3-d]pyrimidine-6-carboxamide

Into a 10-mL sealed tube were added dioxane (4 mL), 2-chloro-7-(4-((dimethylamino)methyl)-3-(trifluoromethyl)phenyl)-N,N-dimethyl-7H-pyrrolo[2,3-d]pyrimidine-6-carboxamide (100 mg, 0.23 mmol), (3S,4R)-4-aminotetrahydro-2H-pyran-3-ol hydrochloride (53.9 mg, 0.35 mmol), Pd-PEPPSI-IPentCl 2-methylpyridine-o-picoline (20 mg, 0.023 mmol) and Cs₂CO₃ (229 mg, 0.72 mmol) at room temperature. The resulting mixture was stirred overnight at 100° C. The crude product (30 mg) was purified by Prep-HPLC to give the title product (5.4 mg). ¹H NMR (300 MHz, CD₃OD) δ 8.66 (s, 1H), 7.95-7.83 (m, 2H), 7.63-7.56 (m, 1H), 6.88 (s, 1H), 3.95-3.83 (m, 3H), 3.67 (s, 2H), 3.64-3.50 (m, 1H), 3.48-3.40 (m, 1H), 3.15-2.95 (m, 7H), 2.31 (s, 6H), 2.19-2.13 (m, 1H), 1.85-1.63 (m, 1H). MS (ESI, m/e) [M+H]⁺ 507.4.

### Example 8: 2-(((3S,4R)-3-hydroxytetrahydro-2H-pyran-4-yl)amino)-N,N-dimethyl-7-(2-methyl-1,2,3,4-tetrahydroisoquinolin-6-yl)-7H-pyrrolo[2,3-d]pyrimidine-6-carboxamide

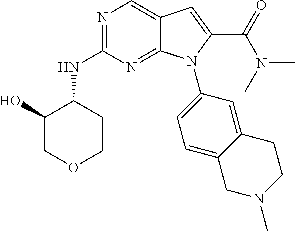 [SMILES: CN1CCc2cc(-n3c(C(=O)N(C)C)cc4cnc(N[C@@H]5CCOC[C@H]5O)nc43)ccc2C1]

### Synthetic Route

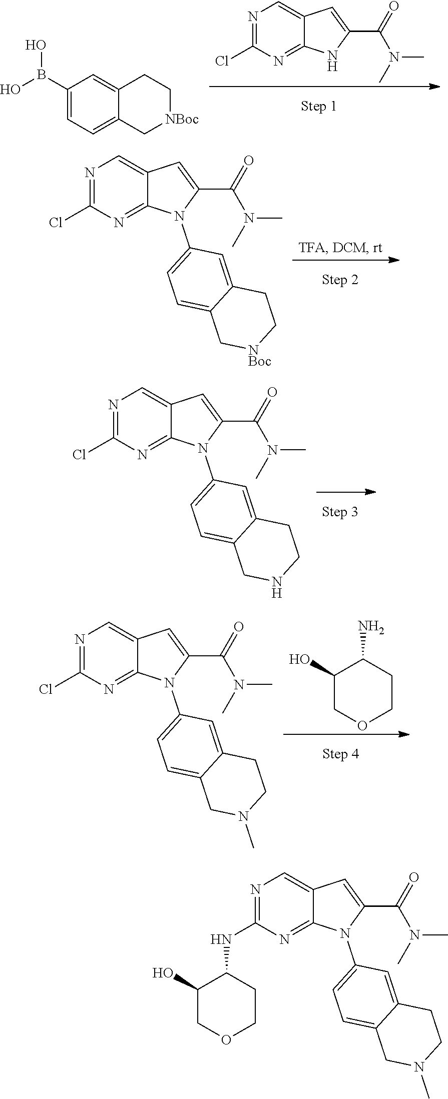

### Step 1: tert-butyl 6-(2-chloro-6-(dimethylcarbamoyl)-7H-pyrrolo[2,3-d]pyrimidin-7-yl)-3,4-dihydroisoquinoline-2(1H)-carboxylate

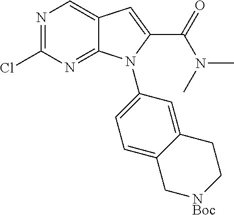 [SMILES: CN(C)C(=O)c1cc2cnc(Cl)nc2n1-c1ccc2c(c1)CCN(C(=O)OC(C)(C)C)C2]

To a stirred solution of 2-chloro-N,N-dimethyl-7H-pyrrolo[2,3-d]pyrimidine-6-carboxamide (0.56 g, 2.00 mmol) and (2-(tert-butoxycarbonyl)-1,2,3,4-tetrahydroisoquinolin-6-yl)boronic acid (0.30 g, 1.34 mmol) in 1,4-dioxane (9 mL) was added Cu(OAc)₂ (0.24 g, 1.33 mmol), pyridine (0.32 g, 4.0 mmol) and 4A MS (0.3 g) at rt. The resulting mixture was stirred for 4 hrs at 80° C. under oxygen atmosphere. The resulting mixture was extracted with EtOAc (2×100 mL). The combined organic layers were washed with brine (2×100 mL), dried over anhydrous Na₂SO₄. After filtration, the filtrate was concentrated under reduced pressure. The resulting mixture was concentrated under reduced pressure. The residue was purified by Prep-TLC (PE/EA=1:1) to give the title product (0.1 g). MS (ESI, m/e) [M+H]⁺ 456.1.

### Step 2: 2-chloro-N,N-dimethyl-7-(1,2,3,4-tetrahydroisoquinolin-6-yl)-7H-pyrrolo[2,3-d]pyrimidine-6-carboxamide

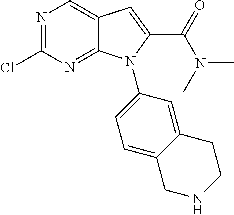 [SMILES: CN(C)C(=O)c1cc2cnc(Cl)nc2n1-c1ccc2c(c1)CCNC2]

To a solution of tert-butyl 6-(2-chloro-6-(dimethylcarbamoyl)-7H-pyrrolo[2,3-d]pyrimidin-7-yl)-3,4-dihydroisoquinoline-2(1H)-carboxylate (100 mg, 0.22 mmol) in DCM (2 mL) was added TFA (1 ml) dropwise at rt. The reaction mixture was stirred at rt for 1 h. The resulting mixture was concentrated under reduced pressure. The residue was purified by Prep-TLC (PE/EA=1:1) to give the title product (50 mg). MS (ESI, m/e) [M+H]⁺ 356.2.

### Step 3: 2-chloro-N,N-dimethyl-7-(2-methyl-1,2,3,4-tetrahydroisoquinolin-6-yl)-7H-pyrrolo[2,3-d]pyrimidine-6-carboxamide

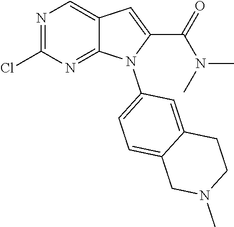 [SMILES: CN1CCc2cc(-n3c(C(=O)N(C)C)cc4cnc(Cl)nc43)ccc2C1]

To a solution of 2-chloro-N,N-dimethyl-7-(1,2,3,4-tetrahydroisoquinolin-6-yl)-7H-pyrrolo[2,3-d]pyrimidine-6-carboxamide (50 mg, 0.14 mmol) in DCE (4 mL) was added formaldehyde (34 mg, 40 wt % in water) dropwise at rt. The reaction mixture was stirred at rt for 2 hrs. To the above mixture was added STAB (48 mg, 0.226 mmol) in portions at 0° C. The resulting mixture was stirred at rt overnight. The resulting mixture was concentrated under reduced pressure. The residue was purified by Prep-TCL to give the title product (13 mg). MS (ESI, m/e) [M+H]⁺ 370.2.

### Step 4: 2-(((3S,4R)-3-hydroxytetrahydro-2H-pyran-4-yl)amino)-N,N-dimethyl-7-(2-methyl-1,2,3,4-tetrahydroisoquinolin-6-yl)-7H-pyrrolo [2,3-d]pyrimidine-6-carboxamide

To a stirred solution of 2-chloro-N,N-dimethyl-7-(2-methyl-1,2,3,4-tetrahydroisoquinolin-6-yl)-7H-pyrrolo[2,3-d]pyrimidine-6-carboxamide (13 mg, 0.04 mmol) and (3S,4R)-4-aminotetrahydro-2H-pyran-3-ol (6.00 mg, 0.04 mmol) in 1,4-dioxane (1 mL) was added Cs₂CO₃ (40 mg, 0.12 mmol) and Pd-PEPPSI-IPentCl 2-methylpyridine (o-picoline) (3.00 mg, 0.004 mmol) in portions at rt. The resulting mixture was stirred for 3 hrs at 100° C. The residue was purified by Prep-HPLC to give the title product (6.1 mg). ¹H NMR (400 MHz, CD₃OD) δ 8.78 (s, 1H), 7.54-7.45 (m, 1H), 7.44-7.33 (m, 2H), 7.00 (s, 1H), 4.80-4.37 (m, 2H), 4.01-3.63 (m, 5H), 3.41 (m, 1H), 3.24-3.13 (m, 7H), 3.00 (s, 3H), 2.11-2.03 (m, 1H), 1.81-1.64 (m, 1H). MS (ESI, m/e) [M+H]⁺ 451.2.

### Example 9: 7-(5-(dimethylamino)-5,6,7,8-tetrahydronaphthalen-2-yl)-2-(((3S,4R)-3-hydroxytetrahydro-2H-pyran-4-yl)amino)-N,N-dimethyl-7H-pyrrolo[2,3-d]pyrimidine-6-carboxamide

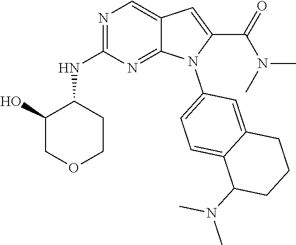 [SMILES: CN(C)C(=O)c1cc2cnc(N[C@@H]3CCOC[C@H]3O)nc2n1-c1ccc2c(c1)CCCC2N(C)C]

### Synthetic Route

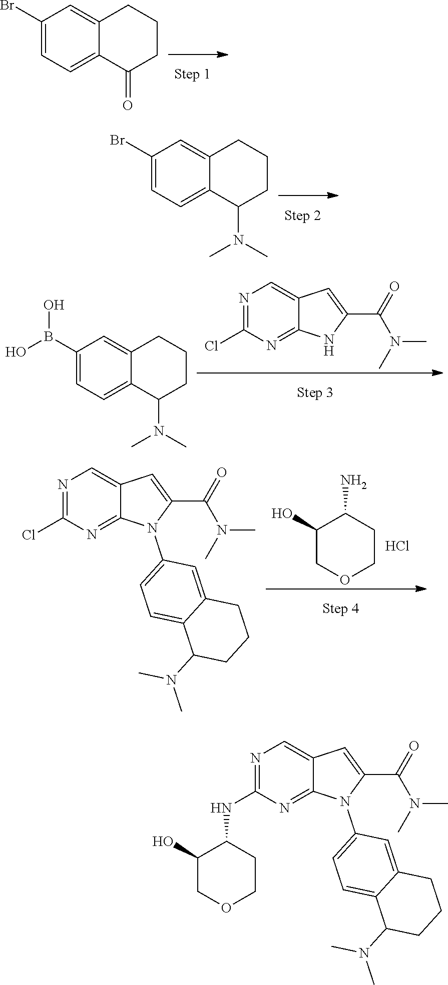

### Step 1: 6-bromo-N,N-dimethyl-1,2,3,4-tetrahydronaphthalen-1-amine

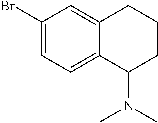

To a mixture of 6-bromo-3,4-dihydronaphthalen-1(2H)-one (2.3 g, 10 mmol), THF (30 mL), and dimethylamine (28.8 ml, 51.8 mmol, 2M in THF) was added TiCl₄ (21.2 mL, 21.2 mmol, 1M in DCM) at 0˜5° C. The resulting solution was stirred for 2 hrs at the same temperature. Then, STAB (6.95 g, 31.0 mmol) was added at 0° C. and the resulting mixture was stirred at room temperature. The mixture was quenched by the addition of 100 mL of water and pH was adjusted to 8 with NaHCO₃. The resulting mixture was extracted by EtOAc and combined organic phase was concentrated. The residue was purified by reverse phase HPLC to give the title product (1.1 g). MS (ESI, m/e) [M+H]⁺ 254.3.

### Step 2: (5-(dimethylamino)-5,6,7,8-tetrahydronaphthalen-2-yl)boronic Acid

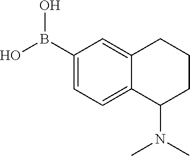 [SMILES: CN(C)C1CCCc2cc(B(O)O)ccc21]

To a solution of 6-bromo-N,N-dimethyl-1,2,3,4-tetrahydronaphthalen-1-amine (0.80 g, 3.2 mmol) in THF (10 mL) was added dropwise n-butyllithium solution (1.64 mL, 4.12 mmol, 2.5M in hexane) at −78° C. The reaction mixture was stirred at −78° C. for 60 min. Then, a solution of triisopropyl borate (0.95 g, 5.1 mmol) in 2 mL THF was added dropwise and the mixture was stirred for another 1 h at room temperature. The reaction was quenched with MeOH (50 mL). The combined organic phase was concentrated under vacuum, and the residue was purified by reverse phase HPLC to give the title product (0.50 g). MS (ESI, m/e) [M+H]⁺ 220.0.

### Step 3: 2-chloro-7-(5-(dimethylamino)-5,6,7,8-tetrahydronaphthalen-2-yl)-N,N-dimethyl-7H-pyrrolo [2,3-d]pyrimidine-6-carboxamide

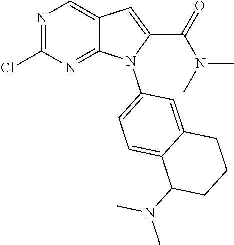 [SMILES: CN(C)C(=O)c1cc2cnc(Cl)nc2n1-c1ccc2c(c1)CCCC2N(C)C]

Into a 10 mL vial were added dioxane (4 mL), (5-(dimethylamino)-5,6,7,8-tetrahydronaphthalen-2-yl)boronic acid (0.50 g, 2.3 mmol), 2-chloro-N,N-dimethyl-7H-pyrrolo[2,3-d]pyrimidine-6-carboxamide (0.51 g, 2.3 mmol), TEA (0.69 g, 6.8 mmol) and Cu(OAc)₂ (0.62 g, 3.4 mmol) at room temperature. The resulting mixture was stirred for 4 hrs at 80° C. under oxygen atmosphere. The residue was purified by Prep-TLC to give the title product. MS (ESI, m/e) [M+H]⁺ 398.2.

### Step 4: 7-(5-(dimethylamino)-5,6,7,8-tetrahydronaphthalen-2-yl)-2-(((3S,4R)-3-hydroxytetrahydro-2H-pyran-4-yl)amino)-N,N-dimethyl-7H-pyrrolo [2,3-d]pyrimidine-6-carboxamide

Into a 10 mL sealed tube were added dioxane (4 mL), 2-chloro-7-(5-(dimethylamino)-5,6,7,8-tetrahydronaphthalen-2-yl)-N,N-dimethyl-7H-pyrrolo[2,3-d]pyrimidine-6-carboxamide (100 mg, 0.25 mmol), (3S,4R)-4-aminotetrahydro-2H-pyran-3-ol hydrochloride (38.5 mg, 0.25 mmol), Pd-PEPPSI-IPentCl 2-methylpyridine o-picoline (21.1 mg, 0.025 mmol) and Cs₂CO₃ (245.7 mg, 0.75 mmol) at room temperature. The resulting mixture was stirred for overnight at 100° C. Solvents were removed and the crude product was purified by Prep-HPLC to give the title product (4.2 mg). ¹H NMR (300 MHz, CD₃OD) δ 8.63 (s, 1H), 7.68-7.60 (m, 1H), 7.24-7.13 (m, 2H), 6.78 (s, 1H), 3.94-3.80 (m, 4H), 3.61-3.52 (m, 1H), 3.48-3.37 (m, 1H), 3.19-3.12 (m, 1H), 3.03-2.92 (m, 6H), 2.89-2.74 (m, 2H), 2.30 (s, 6H), 2.22-1.55 (m, 6H). MS (ESI, m/e) [M+H]⁺ 479.4.

### Example 10: 7-(4′-(dimethylamino)-3′,4′-dihydro-2′H-spiro[cyclopropane-1,1′-naphthalen]-7′-yl)-2-(((3S,4R)-3-hydroxytetrahydro-2H-pyran-4-yl)amino)-N,N-dimethyl-7H-pyrrolo[2,3-d]pyrimidine-6-carboxamide

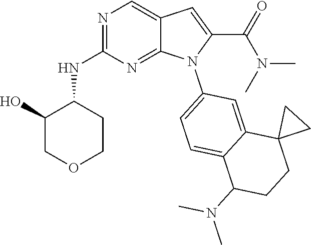 [SMILES: CN(C)C(=O)c1cc2cnc(N[C@@H]3CCOC[C@H]3O)nc2n1-c1ccc2c(c1)C1(CCC2N(C)C)CC1]

### Synthetic Route

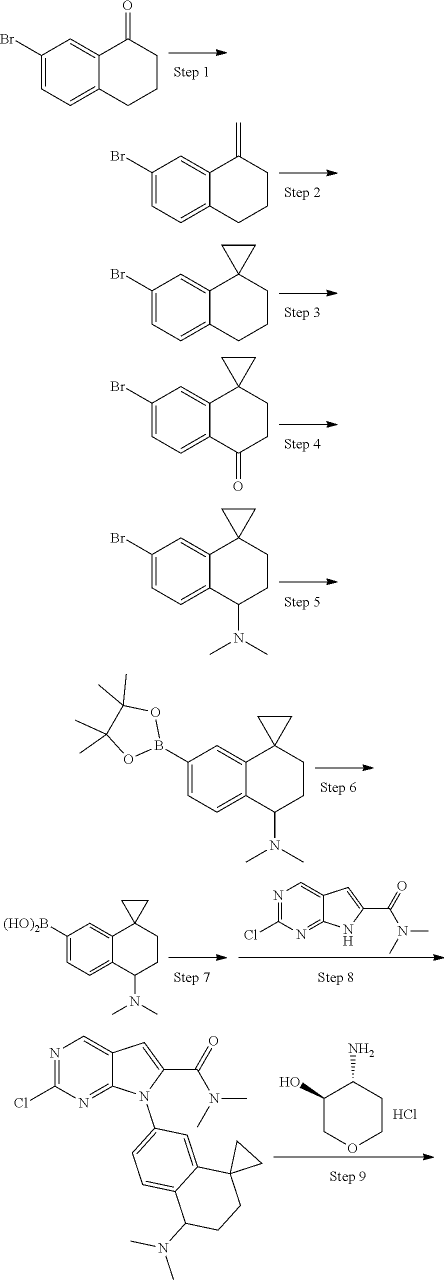

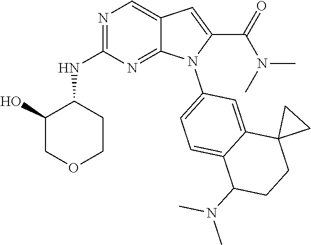 [SMILES: CN(C)C(=O)c1cc2cnc(N[C@@H]3CCOC[C@H]3O)nc2n1-c1ccc2c(c1)C1(CCC2N(C)C)CC1]

### Step 1: 7-bromo-1-methylene-1,2,3,4-tetrahydronaphthalene

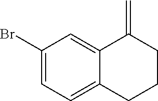

A mixture of bromo(methyl)triphenyl-lambda5-phosphane (4.86 g, 13.6 mmol) and t-BuOK (2.30 g, 20.5 mmol) in toluene was stirred for 40 min at 110° C. To the above mixture was added 7-bromo-3,4-dihydro-2H-naphthalen-1-one (3 g, 13.33 mmol). The resulting mixture was stirred for additional 15 min at 110° C. The resulting mixture was extracted with EtOAc (3×100 mL). The combined organic layers were washed with brine (2×200 mL), and dried over anhydrous Na₂SO₄. After filtration, the filtrate was concentrated under reduced pressure. The residue was purified by silica gel column chromatography to afford the title product (1.7 g).

### Step 2: 7′-bromo-3′,4′-dihydro-2′H-spiro[cyclopropane-1,1′-naphthalene]

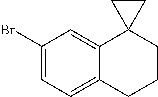 [SMILES: Brc1ccc2c(c1)C1(CCC2)CC1]

To a stirred solution of 7-bromo-1-methylene-1,2,3,4-tetrahydronaphthalene (2.0 g, 9.0 mmol) and chloroiodomethane (6.48 mL, 89.6 mmol) in DCE was added diethylzinc (71.7 mL, 71.7 mmol) in portions at 0° C. The resulting mixture was stirred for 3 hrs at 0° C. The reaction was quenched with sat. NH₄Cl (aq.) at 0° C. The resulting mixture was extracted with EtOAc (3×100 mL). The combined organic layers were washed with brine (200 mL), and dried over anhydrous Na₂SO₄. After filtration, the filtrate was concentrated under reduced pressure. The crude product (1.9 g) was used in the next step directly without further purification.

### Step 3: 7′-bromo-2′,3′-dihydro-4′H-spiro[cyclopropane-1,1′-naphthalen]-4′-one

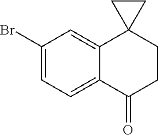 [SMILES: O=C1CCC2(CC2)c2cc(Br)ccc21]

To a stirred solution of 7′-bromo-3′,4′-dihydro-2′H-spiro[cyclopropane-1,1′-naphthalene] (1.9 g, 8.0 mmol) and KMnO₄ (1.90 g, 12.0 mmol) in t-BuOH were added KH₂PO₄ (2.18 g, 16.0 mmol) and Na₂HPO₄ (2.27 g, 16.0 mmol) in portions at room temperature. The resulting mixture was stirred for 2 hrs at room temperature. The resulting mixture was diluted with EtOAc (200 mL). The resulting mixture was washed with 200 mL of brine. The residue was purified by silica gel column chromatography to give the title product (1.5 g). MS (ESI, m/e) [M+H]⁺ 251.1.

### Step 4: 7′-bromo-N,N-dimethyl-3′,4′-dihydro-2′H-spiro[cyclopropane-1,1′-naphthalen]-4′-amine

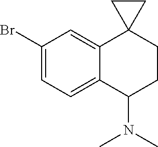 [SMILES: CN(C)C1CCC2(CC2)c2cc(Br)ccc21]

To a stirred solution of 7′-bromo-2′,3′-dihydro-4′H-spiro[cyclopropane-1,1′-naphthalen]-4′-one (1.0 g, 4.0 mmol) and dimethylamine (9.96 mL, 19.9 mmol) in THF was added TiCl₄ (8 mL, 8.0 mmol) in portions at 0° C. The resulting mixture was stirred for 2 hrs at room temperature. To the above mixture was added STAB (1.69 g, 7.96 mmol) at room temperature. The resulting mixture was stirred for additional 2 hrs at room temperature. The reaction was quenched with water at room temperature. The resulting mixture was extracted with EtOAc (3×100 mL). The combined organic layers were washed with brine (300 mL), and dried over anhydrous Na₂SO₄. After filtration, the filtrate was concentrated under reduced pressure. The residue was purified by silica gel column chromatography to give the title product (700 mg). MS (ESI, m/e) [M+H]⁺ 280.2.

### Step 5: N,N-dimethyl-7′-(4,4,5,5-tetramethyl-1,3,2-dioxaborolan-2-yl)-3′,4′-dihydro-2′H-spiro[cyclopropane-1,1′-naphthalen]-4-amine

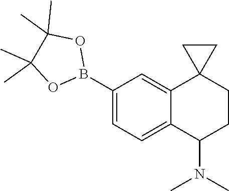

To a mixture of 7′-bromo-N,N-dimethyl-3′,4′-dihydro-2′H-spiro[cyclopropane-1,1′-naphthalen]-4′-amine (550 mg, 1.96 mmol) and bis(pinacolato)diboron (747.6 mg, 2.94 mmol) in dioxane were added KOAc (385.2 mg, 3.92 mmol) and Pd(dppf)Cl₂ (143.6 mg, 0.19 mmol) at room temperature. The resulting mixture was stirred overnight at 80° C. The resulting mixture was extracted with EtOAc (3×60 mL). The combined organic layers were washed with brine (100 mL), and dried over anhydrous Na₂SO₄. After filtration, the filtrate was concentrated under reduced pressure. The residue was purified by silica gel column chromatography to give the title product (450 mg).

### Step 6: (4′-(dimethylamino)-3′,4′-dihydro-2′H-spiro[cyclopropane-1,1′-naphthalen]-7′-yl)boronic Acid

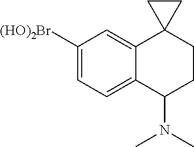

To a mixture of N,N-dimethyl-7′-(4,4,5,5-tetramethyl-1,3,2-dioxaborolan-2-yl)-3′,4′-dihydro-2′H-spiro[cyclopropane-1,1′-naphthalen]-4′-amine (400 mg, 1.22 mmol) and NaIO₄ (784.2 mg, 3.66 mmol) in THF/H₂O was added HCl (0.8 mL, 0.85 mmol) at room temperature. The resulting mixture was stirred for 1 h at room temperature. The mixture was basified to pH 8 with saturated NaHCO₃ (aq.). The resulting mixture was extracted with EtOAc (3×40 mL). The combined organic layers were washed with brine (100 mL), and dried over anhydrous Na₂SO₄. After filtration, the filtrate was concentrated under reduced pressure. The residue was purified by silica gel column chromatography to give the title product (200 mg). MS (ESI, m/e) [M+H]⁺ 246.3.

### Step 7: 2-chloro-7-(4′-(dimethylamino)-3′,4′-dihydro-2′H-spiro[cyclopropane-1,1′-naphthalen]-7′-yl)-N,N-dimethyl-7H-pyrrolo[2,3-d]pyrimidine-6-carboxamide

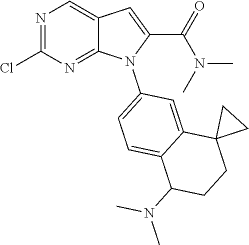 [SMILES: CN(C)C(=O)c1cc2cnc(Cl)nc2n1-c1ccc2c(c1)C1(CCC2N(C)C)CC1]

To a mixture of (4′-(dimethylamino)-3′,4′-dihydro-2′H-spiro[cyclopropane-1,1′-naphthalen]-7′-yl)boronic acid (170 mg, 0.69 mmol) and 2-chloro-N,N-dimethyl-7H-pyrrolo[2,3-d]pyrimidine-6-carboxamide (202.5 mg, 0.90 mmol) in 1,4-dioxane were added pyridine (0.18 mL, 1.4 mmol) and Cu(OAc)₂ (125.9 mg, 0.69 mmol), 4A MS (340 mg) at room temperature. The resulting mixture was stirred overnight at 80° C. under oxygen atmosphere. The resulting mixture was extracted with EtOAc (3×30 mL). The combined organic layers were washed with brine (50 mL), and dried over anhydrous Na₂SO₄. After filtration, the filtrate was concentrated under reduced pressure. The residue was purified by silica gel column chromatography to give the title product (60 mg). MS (ESI, m/e) [M+H]⁺ 424.0.

### Step 8: 7-(4′-(dimethylamino)-3′,4′-dihydro-2′H-spiro[cyclopropane-1,1′-naphthalen]-7′-yl)-2-(((3S,4R)-3-hydroxytetrahydro-2H-pyran-4-yl)amino)-N,N-dimethyl-7H-pyrrolo[2,3-d]pyrimidine-6-carboxamide

To a stirred solution of 2-chloro-7-(4′-(dimethylamino)-3′,4′-dihydro-2′H-spiro[cyclopropane-1,1′-naphthalen]-7′-yl)-N,N-dimethyl-7H-pyrrolo[2,3-d]pyrimidine-6-carboxamide (55 mg, 0.13 mmol) and (3S,4R)-4-aminooxan-3-ol hydrochloride (29.8 mg, 0.19 mmol) in 1,4-dioxane were added Cs₂CO₃ (126.8 mg, 0.39 mmol) and [1,3-bis[2,6-bis(1-ethylpropyl)phenyl]-4,5-dichloro-1,3-dihydro-2H-imidazol-2-ylidene]dichloro(2-methylpyridine)Palladium (10.9 mg, 0.01 mmol) at room temperature. The resulting mixture was stirred overnight at 100° C. The resulting mixture was extracted with EtOAc (3×20 mL). The combined organic layers were washed with brine (30 mL), dried over anhydrous Na₂SO₄. After filtration, the filtrate was concentrated under reduced pressure. The residue was purified by Prep-TLC to give the title product (17 mg). ¹H NMR (300 MHz, CD₃OD) δ 8.75 (s, 1H), 7.63-7.56 (m, 1H), 7.42-7.34 (m, 1H), 7.09-6.94 (m, 2H), 4.01-3.79 (m, 3H), 3.71-3.54 (m, 1H), 3.44-3.34 (m, 2H), 3.21-3.07 (m, 4H), 3.07-2.93 (m, 6H), 2.93-2.72 (m, 3H), 2.44-2.30 (m, 2H), 2.15-1.98 (m, 1H), 1.98-1.83 (m, 2H), 1.81-1.60 (m, 1H), 1.22-0.95 (m, 4H). MS (ESI, m/e) [M+H]⁺ 505.3.

### Example 11: 7-(5-(dimethylamino)-8,8-difluoro-5,6,7,8-tetrahydronaphthalen-2-yl)-2-(((3S,4R)-3-hydroxytetrahydro-2H-pyran-4-yl)amino)-N,N-dimethyl-7H-pyrrolo[2,3-d]pyrimidine-6-carboxamide

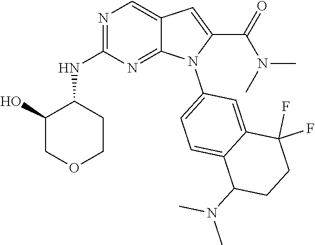 [SMILES: CN(C)C(=O)c1cc2cnc(N[C@@H]3CCOC[C@H]3O)nc2n1-c1ccc2c(c1)C(F)(F)CCC2N(C)C]

### Synthetic Route

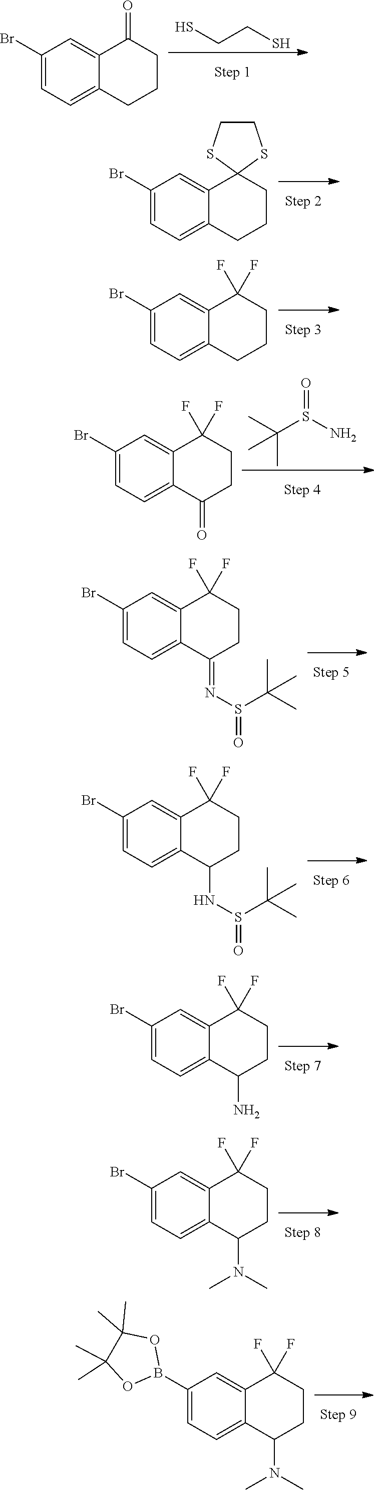

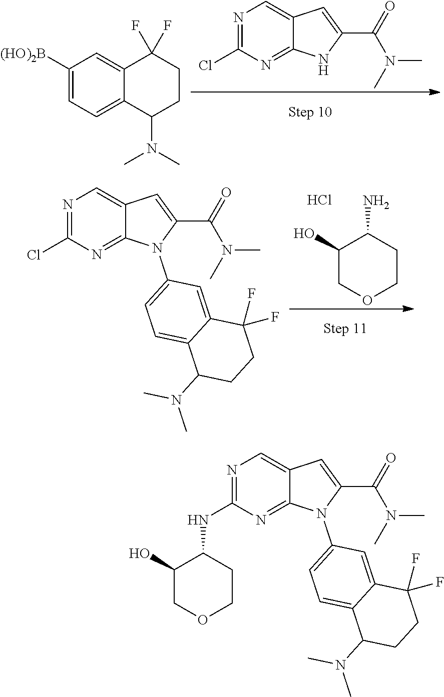

### Step 1: 7-bromo-3,4-dihydro-2H-spiro[naphthalene-1,2′-[1,3]dithiolane]

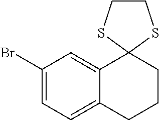 [SMILES: Brc1ccc2c(c1)C1(CCC2)SCCS1]

Into a 150 mL round-bottom flask were added 7-bromo-3,4-dihydro-2H-naphthalen-1-one (11 g, 49 mmol), 1,2-ethanedithiol (9.2 g, 98 mmol), PTSA (0.84 g, 4.9 mmol) and toluene (150 mL) at room temperature. The resulting mixture was stirred overnight at room temperature. The resulting mixture was concentrated under reduced pressure. The residue was purified by silica gel column chromatography to afford the title product (13 g).

### Step 2: 7-bromo-1,1-difluoro-1,2,3,4-tetrahydronaphthalene

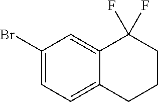 [SMILES: FC1(F)CCCc2ccc(Br)cc21]

A mixture of NIS (38.8 g, 173 mmol) and DCM (30 mL) was cooled to −78 VC. HF-Pyridine (34.2 g, 345 mmol) was added, followed by 7-bromo-3,4-dihydro-2H-spiro[naphthalene-1,2′-[1,3]dithiolane] (13 g, 43.15 mmol). The reaction was stirred at −78° C. for 1 h, and then stirred at 0° C. for additional 12 hrs. The reaction mixture was quenched by NaHCO₃ and extracted with DCM (3*25 mL). The residue was purified by silica gel column chromatography to afford the title product (5.8 g).

### Step 3: 6-bromo-4,4-difluoro-3,4-dihydronaphthalen-1(2H)-one

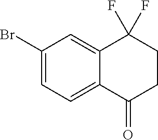 [SMILES: O=C1CCC(F)(F)c2cc(Br)ccc21]

Into a 250-mL round-bottom flask were added 7-bromo-1,1-difluoro-1,2,3,4-tetrahydronaphthalene (5.80 g, 23.5 mmol), KMnO₄ (11.1 g, 70.4 mmol), KH₂PO₄ (12.8 g, 93.9 mmol), Na₂HPO₄ (13.3 g, 93.9 mmol), t-BuOH (60 mL) and H₂O (36 mL) at room temperature. The mixture was stirred overnight at room temperature. The resulting mixture was extracted with EtOAc (2×200 mL). The combined organic layers were washed with brine (2×50 mL), and dried over anhydrous Na₂SO₄. After filtration, the filtrate was concentrated under reduced pressure. The residue was purified by silica gel column chromatography to afford the title product (5.3 g).

### Step 4: (E)-N-(6-bromo-4,4-difluoro-3,4-dihydronaphthalen-1(2H)-ylidene)-2-methylpropane-2-sulfinamide

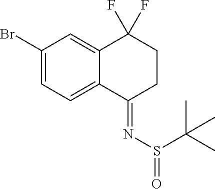 [SMILES: CC(C)(C)S(=O)/N=C1\CCC(F)(F)c2cc(Br)ccc21]

Into a 100-mL round-bottom flask were added 6-bromo-4,4-difluoro-3,4-dihydronaphthalen-1(2H)-one (5.3 g, 20 mmol), 2-methylpropane-2-sulfinamide (4.92 g, 40.6 mmol), Titanium ethoxide (13.9 g, 60.9 mmol) and THF (60 mL) at room temperature. The resulting mixture was stirred for 3 hrs at 80° C. The reaction was quenched by water (10 mL) at room temperature. Solvents were removed and the residue was purified by silica gel column chromatography to afford the title product (5.3 g). MS (ESI, m/e) [M+H]⁺ 364.0.

### Step 5: N-(6-bromo-4,4-difluoro-1,2,3,4-tetrahydronaphthalen-1-yl)-2-methylpropane-2-sulfinamide

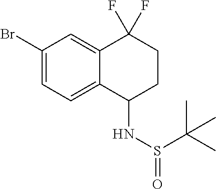 [SMILES: CC(C)(C)S(=O)NC1CCC(F)(F)c2cc(Br)ccc21]

Into a 60-mL round-bottom flask were added (E)-N-(6-bromo-4,4-difluoro-3,4-dihydronaphthalen-1(2H)-ylidene)-2-methylpropane-2-sulfinamide (5.30 g, 14.6 mmol), NaBH₄ (1.10 g, 29.1 mmol) and MeOH (60 mL) at room temperature. The resulting mixture was stirred for 2 hrs at room temperature. The resulting mixture was concentrated under reduced pressure. The residue was purified by silica gel column chromatography to give the title product (5.0 g). MS (ESI, m/e) [M+H]⁺ 366.0.

### Step 6: 6-bromo-4,4-difluoro-1,2,3,4-tetrahydronaphthalen-1-amine

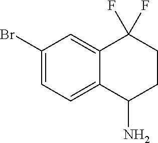 [SMILES: NC1CCC(F)(F)c2cc(Br)ccc21]

Into a 100-mL round-bottom flask were added N-(6-bromo-4,4-difluoro-1,2,3,4-tetrahydronaphthalen-1-yl)-2-methylpropane-2-sulfinamide (5.0 g, 14 mmol) and HCl (gas) in 1,4-dioxane (1.49 g, 40.9 mmol) at room temperature. The resulting mixture was stirred for 3 hrs at room temperature. The resulting mixture was concentrated under reduced pressure. The residue was purified by reverse flash chromatography to give the title product (2.5 g). MS (ESI, m/e) [M+H]⁺ 262.0.

### Step 7: 6-bromo-4,4-difluoro-N,N-dimethyl-1,2,3,4-tetrahydronaphthalen-1-amine

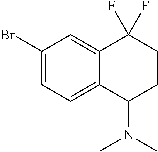 [SMILES: CN(C)C1CCC(F)(F)c2cc(Br)ccc21]

Into a 50-mL round-bottom flask were added 6-bromo-4,4-difluoro-1,2,3,4-tetrahydronaphthalen-1-amine (2.4 g, 9.2 mmol), NaOAc (0.75 g, 9.2 mmol), AcOH (2.75 g, 45.8 mmol), formaldehyde (2.75 g, 91.6 mmol) and DCE (30 mL) at room temperature. The mixture was acidified to pH 8-10 with saturated NaHCO₃ (aq.). The resulting mixture was stirred at 50° C. To the above mixture was added STAB (5.82 g, 27.5 mmol) in portions at room temperature. The resulting mixture was stirred for additional 3 hrs at room temperature. The resulting mixture was concentrated under reduced pressure. The residue was purified by reverse flash chromatography to give the title product (1.2 g). MS (ESI, m/e) [M+H]⁺ 289.9.

### Step 8: 4,4-difluoro-N,N-dimethyl-6-(4,4,5,5-tetramethyl-1,3,2-dioxaborolan-2-yl)-1,2,3,4-tetrahydronaphthalen-1-amine

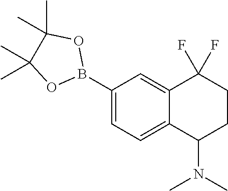

Into a 50-mL round-bottom flask were added 6-bromo-4,4-difluoro-N,N-dimethyl-1,2,3,4-tetrahydronaphthalen-1-amine (1.2 g, 4.1 mmol), bis(pinacolato)diboron (2.10 g, 8.27 mmol), Pd(dppf)Cl₂ (0.30 g, 0.41 mmol), KOAc (0.81 g, 8.3 mmol) and dioxane (15 mL) at room temperature. The reaction mixture was stirred for 2 hrs at 80° C. The resulting mixture was concentrated under reduced pressure. The residue was purified by silica gel column chromatography to give the title product (1 g). MS (ESI, m/e) [M+H]⁺ 338.2.

### Step 9: (5-(dimethylamino)-8,8-difluoro-5,6,7,8-tetrahydronaphthalen-2-yl)boronic Acid

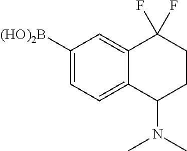 [SMILES: CN(C)C1CCC(F)(F)c2cc(B(O)O)ccc21]

Into a 50-mL round-bottom flask were added 4,4-difluoro-N,N-dimethyl-6-(4,4,5,5-tetramethyl-1,3,2-dioxaborolan-2-yl)-1,2,3,4-tetrahydronaphthalen-1-amine (1.0 g, 2.9 mmol), Sodium periodate (1.89 g, 8.83 mmol), THF (12 mL) and H₂O (3 mL) at room temperature. The resulting mixture was stirred for 2 hrs at room temperature. The residue was purified by reverse flash chromatography to give the title product (300 mg). MS (ESI, m/e) [M+H]⁺ 256.1.

### Step 10: 2-chloro-7-(5-(dimethylamino)-8,8-difluoro-5,6,7,8-tetrahydronaphthalen-2-yl)-N,N-dimethyl-7H-pyrrolo[2,3-d]pyrimidine-6-carboxamide

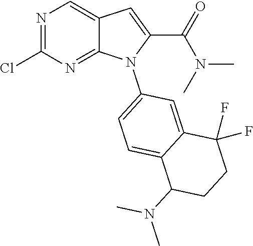 [SMILES: CN(C)C(=O)c1cc2cnc(Cl)nc2n1-c1ccc2c(c1)C(F)(F)CCC2N(C)C]

A mixture of (5-(dimethylamino)-8,8-difluoro-5,6,7,8-tetrahydronaphthalen-2-yl)boronic acid (300 mg, 1.17 mmol), 2-chloro-N,N-dimethyl-7H-pyrrolo[2,3-d]pyrimidine-6-carboxamide (176 mg, 0.78 mmol), triethylamine (238 mg, 2.35 mmol), Cu(OAc)₂ (214 mg, 1.17 mmol), dioxane (5 mL) and 4A MS (100 mg) was stirred at 80° C. overnight under oxygen atmosphere. The mixture was filtered and the filtrate was concentrated. The residue was purified by Prep-TLC to give the title product (52 mg). MS (ESI, m/e) [M+H]⁺ 434.0.

### Step 11: 7-(5-(dimethylamino)-8,8-difluoro-5,6,7,8-tetrahydronaphthalen-2-yl)-2-(((3S,4R)-3-hydroxytetrahydro-2H-pyran-4-yl)amino)-N,N-dimethyl-7H-pyrrolo[2,3-d]pyrimidine-6-carboxamide

A mixture of 2-chloro-7-(5-(dimethylamino)-8,8-difluoro-5,6,7,8-tetrahydronaphthalen-2-yl)-N,N-dimethyl-7H-pyrrolo[2,3-d]pyrimidine-6-carboxamide (100 mg, 0.23 mmol), (3S,4R)-4-aminotetrahydro-2H-pyran-3-ol hydrochloride (53 mg, 0.34 mmol), Cs₂CO₃ (17.5 mg, 0.23 mmol), [1,3-bis[2,6-bis(1-ethylpropyl)phenyl]-4,5-dichloro-1,3-dihydro-2H-imidazol-2-ylidene]dichloro(2-methylpyridine)Palladium (19.3 mg, 0.02 mmol) and dioxane (5 mL) was stirred overnight at 100° C. The resulting mixture was concentrated. The residue was purified by Prep-HPLC to afford the title product (5.4 mg). ¹H NMR (300 MHz, CD₃OD) δ 8.77 (s, 1H), 8.13-8.01 (m, 1H), 7.86-7.77 (m, 1H), 7.76-7.64 (m, 1H), 7.05 (s, 1H), 5.08-4.97 (m, 1H), 4.01-3.80 (m, 3H), 3.73-3.54 (m, 1H), 3.44-3.34 (m, 1H), 3.24 (s, 3H), 3.15-2.80 (m, 10H), 2.68-2.36 (m, 4H), 2.13-2.02 (m, 1H), 1.74-1.58 (m, 1H). MS (ESI, m/e) [M+H]⁺ 515.1.

### Example 12: 7-(1-(dimethylamino)-2,3-dihydro-1H-inden-5-yl)-2-(((3S,4R)-3-hydroxytetrahydro-2H-pyran-4-yl)amino)-N,N-dimethyl-7H-pyrrolo[2,3-d]pyrimidine-6-carboxamide

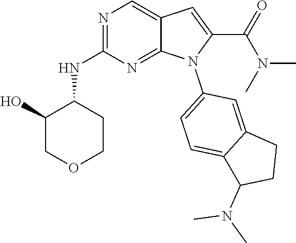 [SMILES: CN(C)C(=O)c1cc2cnc(N[C@@H]3CCOC[C@H]3O)nc2n1-c1ccc2c(c1)CCC2N(C)C]

### Synthetic Route

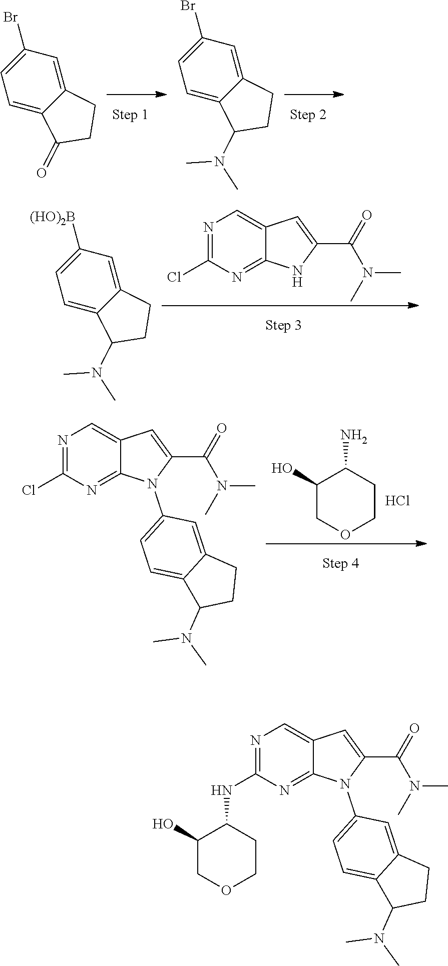

### Step 1: 5-bromo-N,N-dimethyl-2,3-dihydro-1H-inden-1-amine

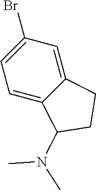

To a mixture of 5-bromo-2,3-dihydro-1H-inden-1-one (5.0 g, 24 mmol), THF (120 mL), dimethylamine (59.5 mL, 119 mmol, 2M in THF) was added TiCl₄ (47.6 mL, 47.6 mmol, 1 M in DCM) at 0˜5° C. The resulting solution was stirred for 2 hrs at the same temperature. This was followed by the addition of STAB (15.1 g, 71.4 mmol) at 0° C. The resulting solution was stirred overnight at room temperature. The mixture was quenched by the addition of water (100 mL) and adjusted pH to 8 with NaHCO₃. The resulting mixture was extracted by EtOAc. Combined organic layer was concentrated. The residue was purified by reverse phase HPLC to give the title product (3.5 g). MS (ESI, m/e) [M+H]⁺ 240.1.

### Step 2: (1-(dimethylamino)-2,3-dihydro-1H-inden-5-yl)boronic Acid

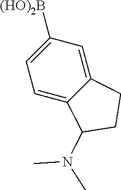

To a mixture of 5-bromo-N,N-dimethyl-2,3-dihydro-1H-inden-1-amine (3.50 g, 14.6 mmol) in THF (40 mL) was added n-butyl lithium (11.7 mL, 29.3 mmol, 2.5M in THF) at −78° C. The mixture was stirred for 1 h at the same temperature. Then, triisopropyl borate (44 g, 23 mmol) was added at −78° C. The resulting solution was stirred overnight at room temperature. The mixture was quenched by MeOH (20 mL) at 0° C. The resulting mixture was concentrated. The residue was purified by reverse phase HPLC to give the title product (1.6 g). MS (ESI, m/e) [M+H]⁺ 206.1.

### Step 3: 2-chloro-7-(1-(dimethylamino)-2,3-dihydro-1H-inden-5-yl)-N,N-dimethyl-7H-pyrrolo[2,3-d]pyrimidine-6-carboxamide

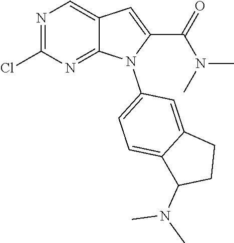 [SMILES: CN(C)C(=O)c1cc2cnc(Cl)nc2n1-c1ccc2c(c1)CCC2N(C)C]

A mixture of (1-(dimethylamino)-2,3-dihydro-1H-inden-5-yl)boronic acid (1.5 g, 7.3 mmol), TEA (2.22 g, 22.0 mmol), Copper acetate (1.99 g, 10.9 mmol), dioxane (15 mL) and 4A MS (1.5 g), 2-chloro-N,N-dimethyl-7H-pyrrolo[2,3-d]pyrimidine-6-carboxamide (1.64 g, 7.3 mmol) was stirred under oxygen atmosphere for 2 hrs at 80° C. The mixture was filtered and the filtrate was concentrated. The residue was purified by Prep-TLC to give the title product (300 mg). MS (ESI, m/e) [M+H]⁺ 384.0.

### Step 4: 7-(1-(dimethylamino)-2,3-dihydro-1H-inden-5-yl)-2-(((3S,4R)-3-hydroxytetrahydro-2H-pyran-4-yl)amino)-N,N-dimethyl-7H-pyrrolo[2,3-d]pyrimidine-6-carboxamide

A mixture of 2-chloro-7-(1-(dimethylamino)-2,3-dihydro-1H-inden-5-yl)-N,N-dimethyl-7H-pyrrolo[2,3-d]pyrimidine-6-carboxamide (300 mg, 0.78 mmol), (3S,4R)-4-aminotetrahydro-2H-pyran-3-ol hydrochloride (179 mg, 1.17 mmol), Cs₂CO₃ (764 mg, 2.34 mmol), [1,3-bis[2,6-bis(1-ethylpropyl)phenyl]-4,5-dichloro-1,3-dihydro-2H-imidazol-2-ylidene]dichloro(2-methylpyridine)Palladium (67 mg, 0.08 mmol) and dioxane (5 mL) was stirred overnight at 90° C. The resulting mixture was concentrated. The residue was purified by silica gel column to give the title product (70 mg). ¹H NMR (300 MHz, CD₃OD) δ 8.64 (s, 1H), 7.55-7.47 (m, 1H), 7.40-7.33 (m, 1H), 7.29-7.20 (m, 1H), 6.79 (s, 1H), 4.53-4.40 (m, 1H), 3.99-3.72 (m, 3H), 3.60-3.52 (m. 1H), 3.45-3.36 (m, 1H), 3.19-2.81 (m, 9H), 2.32 (s, 6H), 2.27-2.10 (m, 3H), 1.66-1.52 (m, 1H). MS (ESI, m/e) [M+H]⁺ 465.2.

### Example 13: 7-(3′-(dimethylamino)-2′,3′-dihydrospiro[cyclopropane-1,1′-inden]-6′-yl)-2-(((3S,4R)-3-hydroxytetrahydro-2H-pyran-4-yl)amino)-N,N-dimethyl-7H-pyrrolo[2,3-d]pyrimidine-6-carboxamide

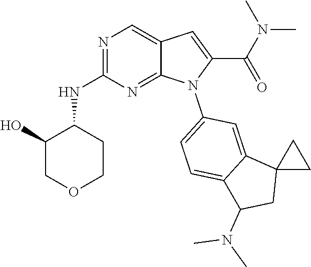 [SMILES: CN(C)C(=O)c1cc2cnc(N[C@@H]3CCOC[C@H]3O)nc2n1-c1ccc2c(c1)C1(CC1)CC2N(C)C]

### Synthetic Route

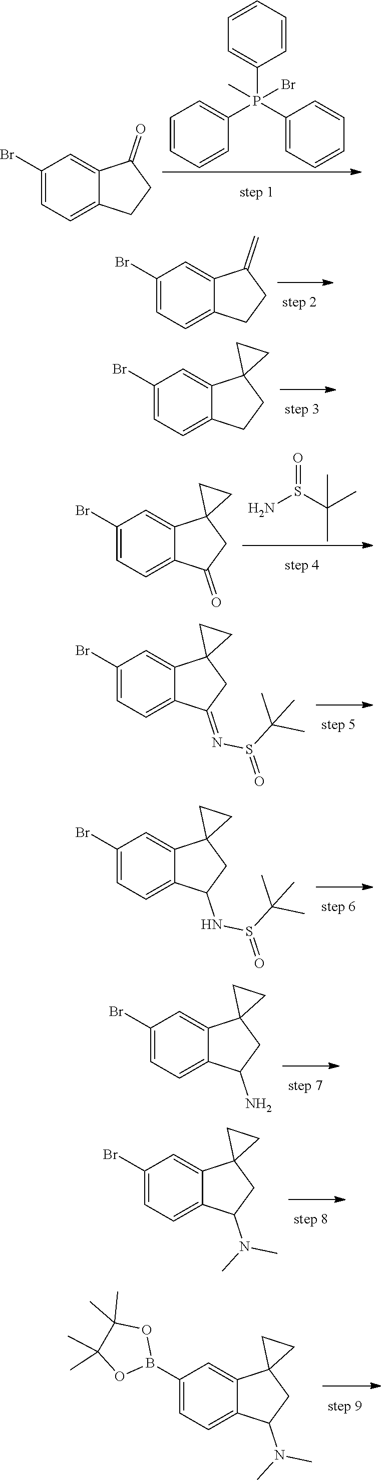

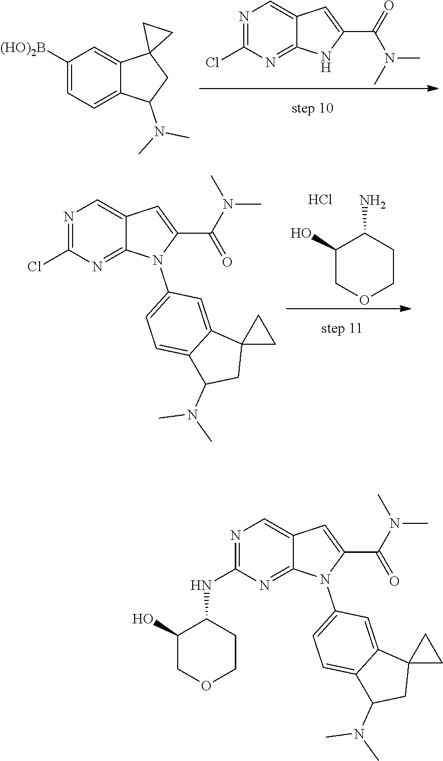

### Step 1: 6-bromo-1-methylene-2,3-dihydro-1H-indene

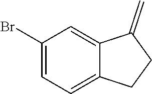

To a stirred solution of 6-bromo-2,3-dihydro-1H-inden-1-one (5.0 g, 24 mmol) and bromo(methyl)triphenyl-lambda5-phosphane (16.9 g, 47.4 mmol) in THF (100 ml) was added t-BuOK (47.6 mL, 47.6 mmol) in portions at room temperature. The resulting mixture was stirred for 3 hrs at room temperature. The resulting mixture was extracted with EtOAc (3×100 mL). The combined organic layers were washed with brine (2×200 mL), and dried over anhydrous Na₂SO₄. After filtration, the filtrate was concentrated under reduced pressure. The residue was purified by silica gel column chromatography to give the title product (3.7 g).

### Step 2: 6′-bromo-2′,3′-dihydrospiro[cyclopropane-1,1′-indene]

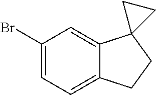 [SMILES: Brc1ccc2c(c1)C1(CC2)CC1]

To a stirred solution of 6-bromo-1-methylene-2,3-dihydro-1H-indene (3.6 g, 17 mmol) and ICH₂Cl (24.2 g, 138 mmol) in DCE (122 mL) was added ZnEt₂ (103 mL, 103.00 mmol) dropwise at 0° C. The mixture was stirred for 3 hrs at room temperature. The reaction was quenched with sat. NH₄Cl (aq.). The resulting mixture was extracted with CH₂Cl₂ (3×150 mL). The combined organic layers were washed with brine (2×300 mL), and dried over anhydrous Na₂SO₄. After filtration, the filtrate was concentrated under reduced pressure. The residue was purified by silica gel column chromatography to give the title product (2.5 g).

### Step 3: 6′-bromospiro[cyclopropane-1,1′-inden]-3′(2′H)-one

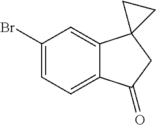 [SMILES: O=C1CC2(CC2)c2cc(Br)ccc21]

To a stirred solution of 6′-bromo-2′,3′-dihydrospiro[cyclopropane-1,1′-indene] (2.0 g, 9.0 mmol) and KH₂PO₄ (2.44 g, 17.9 mmol) in t-BuOH/H₂O (20 mL/10 mL) were added Na₂HPO₄ (2.55 g, 17.9 mmol) and KMnO₄ (2.12 g, 13.4 mmol). The resulting mixture was stirred overnight at room temperature. The resulting mixture was extracted with EtOAc (3×100 mL). The combined organic layers were washed with brine (2×200 mL), and dried over anhydrous Na₂SO₄. After filtration, the filtrate was concentrated under reduced pressure. The residue was purified by silica gel column chromatography to give the title product (1.2 g). MS (ESI, m/e) [M+H]⁺ 236.9.

### Step 4: (E)-N-(6′-bromospiro[cyclopropane-1,1′-inden]-3′(2′H)-ylidene)-2-methylpropane-2-sulfinamide

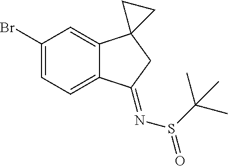 [SMILES: CC(C)(C)S(=O)/N=C1\CC2(CC2)c2cc(Br)ccc21]

To a stirred solution of 6′-bromospiro[cyclopropane-1,1′-inden]-3′(2′H)-one (1.34 g, 5.65 mmol) and 2-methylpropane-2-sulfinamide (1.37 g, 11.3 mmol) in THF (20 ml) was added Ti(OEt)₄ (3.87 g, 17.0 mmol). The resulting mixture was stirred for 4 hrs at 80° C. The reaction was quenched with water. The resulting mixture was extracted with EtOAc (3×100 mL). The combined organic layers were washed with brine (2×200 mL), and dried over anhydrous Na₂SO₄. After filtration, the filtrate was concentrated under reduced pressure. The residue was purified by silica gel column chromatography to give the title product (1.24 g). MS (ESI, m/e) [M+H]⁺ 340.0.

### Step 5: N-(6′-bromo-2′,3′-dihydrospiro[cyclopropane-1,1′-inden]-3′-yl)-2-methylpropane-2-sulfinamide

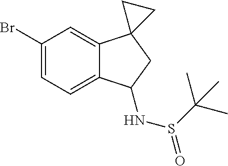 [SMILES: CC(C)(C)S(=O)NC1CC2(CC2)c2cc(Br)ccc21]

A mixture of (E)-N-(6′-bromospiro[cyclopropane-1,1′-inden]-3′(2′H)-ylidene)-2-methylpropane-2-sulfinamide (1.24 g, 3.64 mmol) and NaBH₄ (276 mg, 7.28 mmol) in MeOH (24 mL) was stirred for 2 hrs at room temperature. The reaction was quenched with sat. NH₄Cl (aq.). The resulting mixture was extracted with EtOAc (3×100 mL). The combined organic layers were washed with brine (200 mL), and dried over anhydrous Na₂SO₄. After filtration, the filtrate was concentrated under reduced pressure. The residue was purified by silica gel column chromatography to give the title product (770 mg). MS (ESI, m/e) [M+H]⁺ 342.0.

### Step 6: 6′-bromo-2′,3′-dihydrospiro[cyclopropane-1,1′-inden]-3′-amine

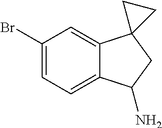 [SMILES: NC1CC2(CC2)c2cc(Br)ccc21]

A solution of N-(6′-bromo-2′,3′-dihydrospiro[cyclopropane-1,1′-inden]-3′-yl)-2-methylpropane-2-sulfinamide (770 mg, 2.24 mmol) and HCl (4 M in 1,4-dioxane) (1.7 mL, 6.74 mmol) in 1,4-dioxane (11.5 mL) was stirred for 30 min at room temperature. The resulting mixture was concentrated under reduced pressure. The crude product was used in the next step directly without further purification.

### Step 7: 6′-bromo-N,N-dimethyl-2′,3′-dihydrospiro[cyclopropane-1,1′-inden]-3′-amine

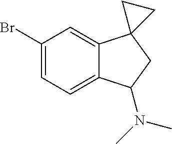

To a mixture of 6′-bromo-2′,3′-dihydrospiro[cyclopropane-1,1′-inden]-3′-amine (550 mg, 2.31 mmol) and NaOAc (189 mg, 2.31 mmol) in DCE (25 mL) were added AcOH (10 mL) and formaldehyde (694 mg, 23.1 mmol). The resulting mixture was stirred for 2 hrs at 50° C. To the above mixture was added STAB (1.47 g, 6.93 mmol) at 50° C. The resulting mixture was stirred for additional 2 hrs at 50° C. The resulting mixture was extracted with EtOAc (3×50 mL). The combined organic layers were washed with brine (100 mL), and dried over anhydrous Na₂SO₄. After filtration, the filtrate was concentrated under reduced pressure. The residue was purified by silica gel column chromatography to give the title product (390 mg). MS (ESI, m/e) [M+H]⁺ 266.0.

### Step 8: N,N-dimethyl-6′-(4,4,5,5-tetramethyl-1,3,2-dioxaborolan-2-yl)-2′,3′-dihydrospiro[cyclopropane-1,1′-inden]-3′-amine

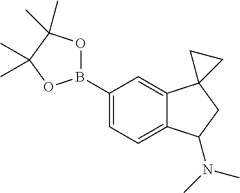

To a mixture of 6′-bromo-N,N-dimethyl-2′,3′-dihydrospiro[cyclopropane-1,1′-inden]-3′-amine (390 mg, 1.46 mmol) and bis(pinacolato)diboron (556 mg, 2.19 mmol) in 1,4-dioxane (6 mL) were added KOAc (287.6 mg, 2.93 mmol) and Pd(dppf)Cl₂ (107 mg, 0.14 mmol). The resulting mixture was stirred for 2 hrs at 80° C. The resulting mixture was extracted with EtOAc (3×50 mL). The combined organic layers were washed with brine (100 mL), and dried over anhydrous Na₂SO₄. After filtration, the filtrate was concentrated under reduced pressure. The residue was purified by Prep-TLC to give the title product (250 mg). MS (ESI, m/e) [M+H]⁺ 314.0.

### Step 9: (3′-(dimethylamino)-2′,3′-dihydrospiro[cyclopropane-1,1′-inden]-6′-yl)boronic Acid

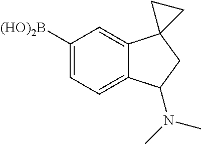 [SMILES: CN(C)C1CC2(CC2)c2cc(B(O)O)ccc21]

To a mixture of N,N-dimethyl-6′-(4,4,5,5-tetramethyl-1,3,2-dioxaborolan-2-yl)-2′,3′-dihydrospiro[cyclopropane-1,1′-inden]-3′-amine (250 mg, 0.79 mmol) and Sodium periodate (512 mg, 2.34 mmol) in THF/H₂O (4 mL/1 mL) was added HCl (1 M, 0.56 mL, 0.55 mmol). The resulting mixture was stirred for 1 h at room temperature. The resulting mixture was concentrated under reduced pressure. The residue was purified by Prep-TLC to give the title product (110 mg). MS (ESI, m/e) [M+H]⁺ 232.0.

### Step 10: 2-chloro-7-(3′-(dimethylamino)-2′,3′-dihydrospiro[cyclopropane-1,1′-inden]-6′-yl)-N,N-dimethyl-7H-pyrrolo[2,3-d]pyrimidine-6-carboxamide

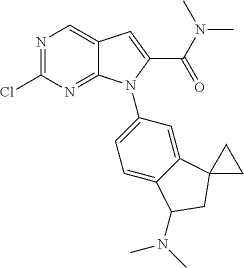 [SMILES: CN(C)C(=O)c1cc2cnc(Cl)nc2n1-c1ccc2c(c1)C1(CC1)CC2N(C)C]

To a stirred solution of (3′-(dimethylamino)-2′,3′-dihydrospiro[cyclopropane-1,1′-inden]-6′-yl)boronic acid (110 mg, 0.47 mmol) and 2-chloro-N,N-dimethyl-7H-pyrrolo[2,3-d]pyrimidine-6-carboxamide (132 mg, 0.58 mmol) in ACN were added Copper acetate (86 mg, 0.47 mmol) and TEA (0.13 ml, 0.95 mmol). To the above mixture was added 4A MS. The resulting mixture was stirred overnight at 80° C. under oxygen atmosphere. The resulting mixture was extracted with EtOAc (3×30 mL). The combined organic layers were washed with brine (60 mL), and dried over anhydrous Na₂SO₄. After filtration, the filtrate was concentrated under reduced pressure. The residue was purified by Prep-TLC to give the title product (70 mg). MS (ESI, m/e) [M+H]⁺ 410.0.

### Step 11: 7-(3′-(dimethylamino)-2′,3′-dihydrospiro[cyclopropane-1,1′-inden]-6′-yl)-2-(((3S,4R)-3-hydroxytetrahydro-2H-pyran-4-yl)amino)-N,N-dimethyl-7H-pyrrolo[2,3-d]pyrimidine-6-carboxamide

To a mixture of 2-chloro-7-(3′-(dimethylamino)-2′,3′-dihydrospiro[cyclopropane-1,1′-inden]-6′-yl)-N,N-dimethyl-7H-pyrrolo[2,3-d]pyrimidine-6-carboxamide (104 mg, 0.25 mmol) and (3S,4R)-4-aminooxan-3-ol hydrochloride (58 mg, 0.38 mmol) in 1,4-dioxane (2.4 mL) were added Cs₂CO₃ (247.6 mg, 0.76 mmol) and [1,3-bis[2,6-bis(1-ethylpropyl)phenyl]-4,5-dichloro-1,3-dihydro-2H-imidazol-2-ylidene]dichloro(2-methylpyridine)Palladium (21.3 mg, 0.03 mmol). The resulting mixture was stirred overnight at 100° C. The resulting mixture was extracted with EtOAc (3×30 mL). The combined organic layers were washed with brine (60 mL), and dried over anhydrous Na₂SO₄. After filtration, the filtrate was concentrated under reduced pressure. The crude product was purified by Prep-HPLC to give the title product (10.9 mg). ¹H NMR (300 MHz, CD₃OD) δ 8.62 (s, 1H), 7.50-7.43 (m, 1H), 7.23-7.18 (m, 1H), 6.87-6.81 (m, 1H), 6.77 (s, 1H), 4.62-4.51 (m, 1H), 3.97-3.77 (m, 3H), 3.61-3.48 (m, 1H), 3.47-3.35 (m, 1H), 3.19-3.08 (m, 1H), 2.93 (s, 6H), 2.38-2.22 (m, 7H), 2.21-2.04 (m, 2H), 1.67-1.51 (m, 1H), 1.16-0.92 (m, 4H). MS (ESI, m/e) [M+H]⁺ 491.3.

### Assays

### Biochemical Assays

### CDK4/Cyclin D1 and CDK6/Cyclin D3 Biochemical Assay

Compounds disclosed herein were tested for inhibition of CDK4/Cyclin D1 or CDK6/Cyclin D3 kinase in an assay based on the time-resolved fluorescence-resonance energy transfer (TR-FRET) methodology. The assay was carried out in 384-well low volume black plates in a reaction mixture containing CDK4/Cyclin D1 or CDK6/Cyclin D3, 1 mM ATP, 0.15 μM Rb (Ser780)-biotin substrate and 0-10 CM compound in buffer containing 50 mM HEPES pH7.0, 0.02% NaN3, 0.01% BSA, 0.1 mM Orthovanadate, 50 mM MgCl2, 1 mM DTT and 0.005% Tween-20. The kinase was incubated with compound for 60 minutes at room temperature and the reaction was initiated by the addition of ATP and Rb (Ser780)-biotin substrate. After reaction at room temperature for 120 minutes, an equal volume of stop/detection solution was added according to the manufacture's instruction (Cisbio Bioassays). The stop/detection solution contained Streptavidin-XL665 and Anti-pRb (Ser780) mAb-Eu Cryptate in Detection buffer (Cisbio Bioassays). Plates were incubated at room temperature for 60 minutes, and the TR-FRET signals (ex337 nm, em665 nm/620 nm) were recorded on a PHERAstar FSX plate reader (BMG Labtech). The inhibition percentage of CDK4/Cyclin D1 or CDK6/Cyclin D3 kinase activity in presence of increasing concentrations of compounds was calculated based on the ratio of fluorescence at 665 nm to that at 620 nm. The IC₅₀ of each compound was derived from fitting the data to the four-parameter logistic equation by Dotmatics.

### Activity Tables

Each of the compounds in Table 1 was tested in one or more of the CDK4 biochemical assays and was found to have activity therein.

TABLE 1

| | | CDK4/Cyclin | CDK6/Cyclin |
|---|---|---|---|
| | | D1 IC50 | D3 IC50 |
| Compound number | Structure | (nM) | (nM) |
| Palbociclib | 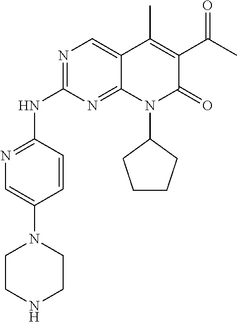 [SMILES: CC(=O)c1c(C)c2cnc(Nc3ccc(N4CCNCC4)cn3)nc2n(C2CCCC2)c1=O] | 10 | 14 |
| Example A94 in WO2019207463 | 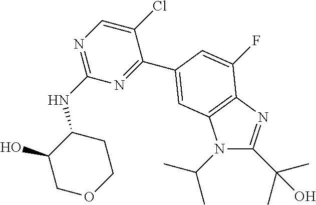 [SMILES: CC(C)n1c(C(C)(C)O)nc2c(F)cc(-c3nc(N[C@@H]4CCOC[C@H]4O)ncc3Cl)cc21] | 5.0 | 120 |
| Example 2 in WO2020212865 | 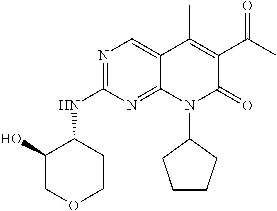 [SMILES: CC(=O)c1c(C)c2cnc(N[C@@H]3CCOC[C@H]3O)nc2n(C2CCCC2)c1=O] | 8.8 | 37 |
| Example 1 | 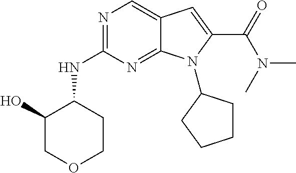 [SMILES: CN(C)C(=O)c1cc2cnc(N[C@@H]3CCOC[C@H]3O)nc2n1C1CCCC1] | 68 | 1041 |
| Example 2 | 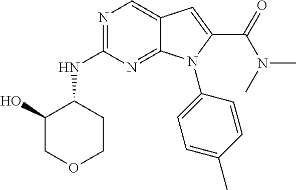 [SMILES: Cc1ccc(-n2c(C(=O)N(C)C)cc3cnc(N[C@@H]4CCOC[C@H]4O)nc32)cc1] | 360 | 8403 |
| Example 3 | 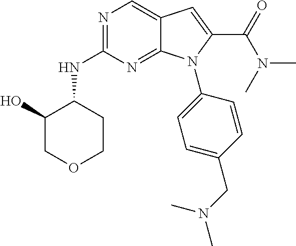 [SMILES: CN(C)Cc1ccc(-n2c(C(=O)N(C)C)cc3cnc(N[C@@H]4CCOC[C@H]4O)nc32)cc1] | 72 | 3540 |
| Example 4 | 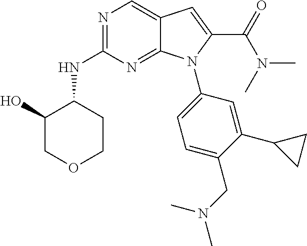 [SMILES: CN(C)Cc1ccc(-n2c(C(=O)N(C)C)cc3cnc(N[C@@H]4CCOC[C@H]4O)nc32)cc1C1CC1] | 9.7 | 778 |
| Example 5 | 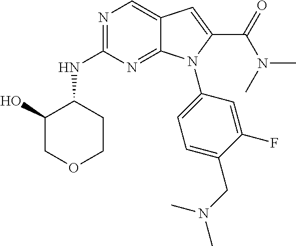 [SMILES: CN(C)Cc1ccc(-n2c(C(=O)N(C)C)cc3cnc(N[C@@H]4CCOC[C@H]4O)nc32)cc1F] | 51 | 5059 |
| Example 6 | 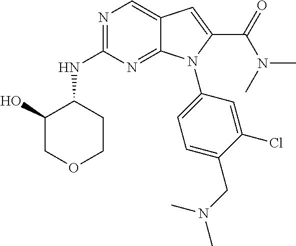 [SMILES: CN(C)Cc1ccc(-n2c(C(=O)N(C)C)cc3cnc(N[C@@H]4CCOC[C@H]4O)nc32)cc1Cl] | 22 | 2772 |
| Example 7 | 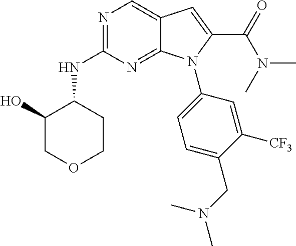 [SMILES: CN(C)Cc1ccc(-n2c(C(=O)N(C)C)cc3cnc(N[C@@H]4CCOC[C@H]4O)nc32)cc1C(F)(F)F] | 5.9 | 710 |
| Example 8 | 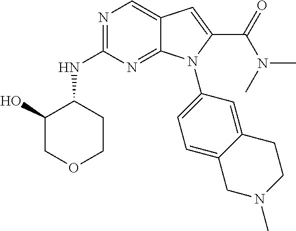 [SMILES: CN1CCc2cc(-n3c(C(=O)N(C)C)cc4cnc(N[C@@H]5CCOC[C@H]5O)nc43)ccc2C1] | 143 | 6759 |
| Example 9 | 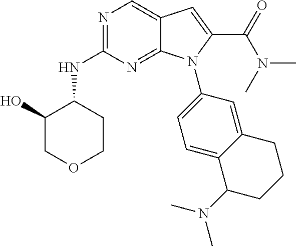 [SMILES: CN(C)C(=O)c1cc2cnc(N[C@@H]3CCOC[C@H]3O)nc2n1-c1ccc2c(c1)CCCC2N(C)C] | 12 | 779 |
| Example 10 | 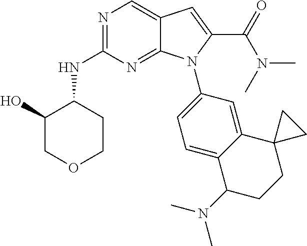 [SMILES: CN(C)C(=O)c1cc2cnc(N[C@@H]3CCOC[C@H]3O)nc2n1-c1ccc2c(c1)C1(CCC2N(C)C)CC1] | 13 | 584 |
| Example 11 | 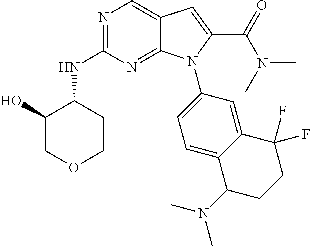 [SMILES: CN(C)C(=O)c1cc2cnc(N[C@@H]3CCOC[C@H]3O)nc2n1-c1ccc2c(c1)C(F)(F)CCC2N(C)C] | 2.1 | 103 |
| Example 12 | 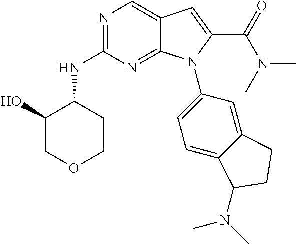 [SMILES: CN(C)C(=O)c1cc2cnc(N[C@@H]3CCOC[C@H]3O)nc2n1-c1ccc2c(c1)CCC2N(C)C] | 42 | 3471 |
| Example 13 | 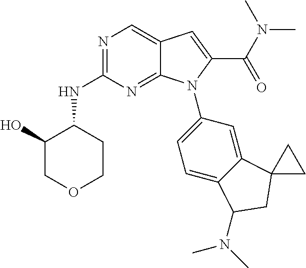 [SMILES: CN(C)C(=O)c1cc2cnc(N[C@@H]3CCOC[C@H]3O)nc2n1-c1ccc2c(c1)C1(CC1)CC2N(C)C] | 38 | 2682 |

It is to be understood that, if any prior art publication is referred to herein; such reference does not constitute an admission that the publication forms a part of the common general knowledge in the art in any country.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various embodiments of the invention.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

A number of references have been cited, the disclosures of which are incorporated herein by reference in their entirety.

What is claimed is:

1. A compound of formula (I): 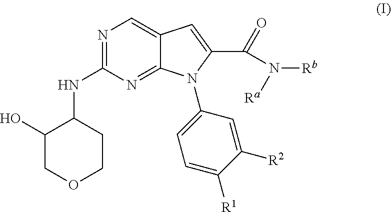
or a pharmaceutically acceptable salt, tautomer, stereoisomer, or enantiomer thereof,
wherein:
each of R^{a} and R^{b} is, independently, hydrogen or substituted or unsubstituted C₁₋₈ alkyl;
or R^{a} and R^{b}, together with the nitrogen to which R^{a} and R^{b} connect, form a substituted or unsubstituted non-aromatic heterocyclyl;
each of R¹ and R² is, independently, hydrogen, halogen, substituted or unsubstituted C₁₋₈ alkyl, substituted or unsubstituted cycloalkyl, substituted or unsubstituted non-aromatic heterocyclyl, substituted or unsubstituted saturated cycloalkylalkyl, substituted or unsubstituted non-aromatic heterocyclylalkyl; or
R¹ and R², together with the atoms to which R¹ and R² connect, form a substituted or unsubstituted cycloalkyl, or substituted or unsubstituted non-aromatic heterocyclyl.

2. The compound of claim 1, wherein the compound is a compound of formula (Ia): 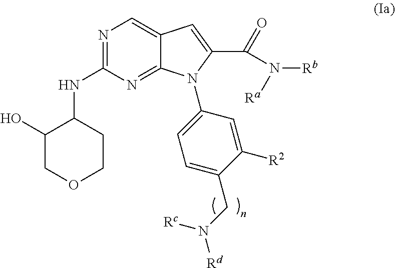
or a pharmaceutically acceptable salt, tautomer, stereoisomer, or enantiomer thereof,
each of R^{c} and R^{d} is, independently, hydrogen or substituted or unsubstituted C₁₋₈ alkyl;
or R^{c} and R^{d}, together with the nitrogen to which R^{c} and R^{d} connect, form a substituted or unsubstituted non-aromatic heterocyclyl; and n is 1, 2 or 3.

3. The compound of claim 1, wherein the compound is a compound of formula (Ib): 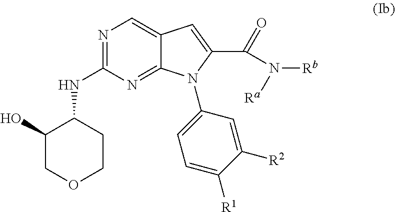
or a pharmaceutically acceptable salt, tautomer, stereoisomer, or enantiomer thereof.

4. The compound of claim 3, wherein the compound is a compound of formula (II): 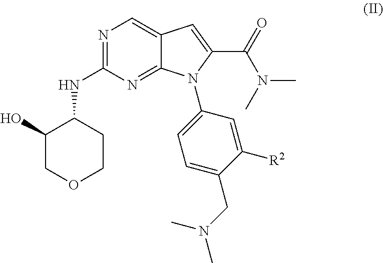
or a pharmaceutically acceptable salt, tautomer, stereoisomer, or enantiomer thereof,
wherein:
R² is hydrogen, halogen, substituted or unsubstituted C₁₋₈ alkyl, or substituted or unsubstituted cycloalkyl.

5. The compound of claim 4, wherein R² is hydrogen, F, Cl, —CF₃, or cyclopropyl.

6. The compound of claim 5, wherein R² is Cl.

7. The compound of claim 5, wherein R² is hydrogen.

8. The compound of claim 5, wherein R² is cyclopropyl.

9. The compound of claim 5, wherein R² is F.

10. The compound of claim 5, wherein R² is —CF₃.

11. The compound of claim 3, wherein the compound is a compound of formula (III): 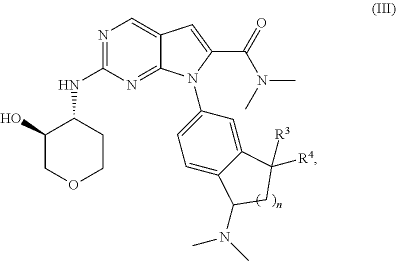
or a pharmaceutically acceptable salt, tautomer, stereoisomer, or enantiomer thereof,
wherein:
each of R³ and R⁴ is, independently, hydrogen, halogen, substituted or unsubstituted C₁₋₈ alkyl; or
R³ and R⁴, together with the atom to which R³ and R⁴ connect, form a substituted or unsubstituted cycloalkyl, or substituted or unsubstituted non-aromatic heterocyclyl; and
n is 1, 2, or 3.

12. The compound of claim 11, wherein R³ and R⁴ are F; and n is 2.

13. The compound of claim 11, wherein R³ and R⁴, together with the atom which R³ and R⁴ connect, form cyclopropyl; and n is 1.

14. The compound of claim 11, wherein R³ and R⁴, together with the atom which R³ and R⁴ connect, form cyclopropyl; and n is 2.

15. The compound of claim 11, wherein R³ and R⁴ are H; and n is 1.

16. The compound of claim 11, wherein R³ and R⁴ are H; and n is 2.

17. The compound of claim 3, wherein the compound is a compound of formula (IV): 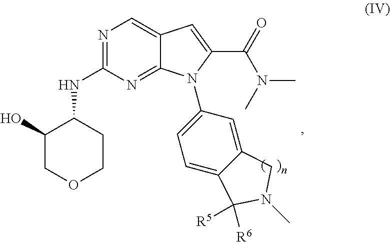
or a pharmaceutically acceptable salt, tautomer, stereoisomer, or enantiomer thereof,
wherein:
each of R⁵ and R⁶ is, independently, hydrogen, halogen, substituted or unsubstituted C₁₋₈ alkyl; or
R⁵ and R⁶, together with the atom to which R⁵ and R⁶ connect, form a substituted or unsubstituted cycloalkyl, or substituted or unsubstituted non-aromatic heterocyclyl; and
n is 1, 2, or 3.

18. The compound of claim 17, wherein R⁵ and R⁶ are H; and n is 2.

19. The compound of claim 3, wherein R¹ is methyl; and R² is H.

20. The compound of any one of claims 1-19, wherein the compound is selected from Table 1.

21. A pharmaceutical composition comprising an effective amount of a compound of any one of claims 1-20, or a pharmaceutically acceptable salt, tautomer, isotopologue, stereoisomer, or prodrug thereof, and a pharmaceutically acceptable carrier, excipient or vehicle.

22. A method of inhibiting activity of cyclin-dependent kinases in a cell, comprising contacting said cell with an effective amount of a compound of any one of claims 1-20, or a pharmaceutically acceptable salt, tautomer, isotopologue, stereoisomer, or prodrug thereof.

23. The method of claim 22, wherein the cyclin-dependent kinase is CDK4.

24. The method of claim 22, wherein the compound is selective for CDK4 over CDK6.

25. The method of claim 24, wherein the compound is selective for CDK4 over CDK1, CDK2, CDK3, CDK5, CDK6, CDK7, CDK8, CDK9, CDK10, or CDK11.

26. The method of claim 24, wherein the compound is at least 5-fold selective for CDK4 over CDK6.

27. The method of claim 24, wherein the compound is at least 50-fold selective for CDK4 over CDK6.

28. The method of claim 24, wherein the compound is at least 100-fold selective for CDK4 over CDK6.

29. A method for the treatment or prevention of CDK4 mediated disorder, the methods comprising administering to a subject in need thereof an effective amount of a compound of any one of claims 1-20.

30. A method for the treatment or prevention of a cancer responsive to CDK4 activity, the methods comprising administering to a subject in need thereof an effective amount of a compound of any one of claims 1-20.
